(19) United States
(12) Patent Application Publication
(10) Pub. No.: US 2026/0271879 A1
(43) Pub. Date: Sep. 17, 2026

(54) System and Method for Real-Time Animal Training and Welfare Management

(71) Applicant: QOMPLX LLC, Reston, VA (US)

(72) Inventors: Jason Crabtree, Vienna, VA (US); Richard Kelley, Woodbridge, VA (US); Jason Hopper, Halifax (CA); David Park, Fairfax, VA (US)

(21) Appl. No.: 19/172,638

(22) Filed: Apr. 7, 2025

Related U.S. Application Data
(63) Continuation-in-part of application No. 19/094,808, filed on Mar. 28, 2025.
Application No. 19/094,808 is a continuation-in-part of application No. 19/078,192, filed on Mar. 12, 2025.

(51) Int. Cl. A01K 15/02; A61B 5/00; A61B 5/16; G16H 10/60; G16H 20/70; G16H 50/30

(52) U.S. Cl. CPC A01K 15/02; A61B 5/165; A61B 5/6803; A61B 5/7264; A61B 5/7405; A61B 5/742; A61B 5/746; A61B 5/7465; G16H 10/60; G16H 20/70; G16H 50/30; A61B 2503/40

(57) ABSTRACT

Disclosed embodiments provide a system and method for adaptive real-time animal training and welfare management, utilizing non-invasive neural monitoring and cognitive assessment via machine learning. The system includes conductive polymer ink electrodes or specialized wearable headgear for continuous acquisition of neural signals. Advanced algorithms perform frequency-domain analysis to correlate specific neural profiles with cognitive states such as attentiveness and stress. A multi-layered machine learning framework interprets these signals, dynamically adjusting training protocols and welfare assessments. Integrated environmental sensors enable multimodal data fusion, refining contextual accuracy. This invention supports continuous data-driven optimization of training efficacy, animal welfare, and predictive performance analytics throughout the animal’s lifecycle.

### CROSS-REFERENCE TO RELATED APPLICATIONS

Priority is claimed in the application data sheet to the following patents or patent applications, each of which is expressly incorporated herein by reference in its entirety:

19/094,808

19/078,192

### BACKGROUND OF THE INVENTION

### Field of the Art

The present invention relates to the field of artificial intelligence enhanced animal training, monitoring, and interaction. More specifically, the invention pertains to systems and methods that enable real-time animal training, enhance veterinary treatment, welfare management and coordination with humans and robots and smart infrastructure.

### Discussion of the State of the Art

Training animals to perform specific tasks can be incredibly beneficial, whether for service, therapy, agriculture, search and rescue, or companionship. Well-trained animals can assist people with disabilities, help locate missing individuals, find bombs or drugs, herd livestock efficiently, and even detect medical conditions. Their contributions enhance human safety, productivity, and well-being. Animals have assisted humans throughout history and will soon be asked to interact with humans, robots and smart infrastructure in new ways. Horses revolutionized transportation, enabling people to travel faster and over greater distances. In the past, horses were also central to warfare, pulling chariots, carrying soldiers, and serving as a vital component of cavalry units. Additionally, mules have been used for hauling heavy loads in mining, agriculture, and construction, especially in rugged terrains where machinery is impractical. Horses significantly shaped human civilization by acting as efficient biological converters of energy, facilitating transportation, agriculture, and warfare. Analyzing their contributions through thermodynamic economic theory highlights how animal-powered labor enhanced economic efficiency and productivity by optimizing energy conversion, resource utilization, and minimizing entropy-related waste.

In a similar manner, oxen have been critical in farming, pulling plows and carts to cultivate large areas of land, especially before the advent of modern machinery. Dogs have been indispensable in hunting, helping track and retrieve game. Dogs also have been used for guarding livestock, property, and even as loyal companions. These partnerships highlight how humans have harnessed the unique abilities of different animals to develop symbiotic relationships between humans and animals. Oxen and dogs have directly contributed to human economies through farming and hunting, serving as specialized biological energy converters and collaborators that optimize productivity and resource management. Dolphins, though not domesticated, have similarly supported human economic efficiency by engaging in cooperative hunting behaviors, enhancing fishing productivity and indirectly optimizing energy use. Thermodynamic economic theory highlights these symbiotic interactions, illustrating how humans have strategically harnessed animal partnerships to increase both tangible resource yields and intangible cultural benefits.

Human-AI-robot integration will serve as the foundation for increased tangible yields and enhanced cultural benefits in the coming era because it creates unprecedented synergies in productivity, efficiency, and innovation. Through intelligent collaboration, AI-driven robots amplify human capabilities by autonomously performing repetitive or precision tasks, optimizing energy and resource use, and generating actionable insights from vast data streams, thus maximizing thermodynamic and economic efficiency. Humans, leveraging their creative, ethical, and strategic strengths, guide this collaborative system toward meaningful goals, resulting in substantial improvements in industrial output, agricultural productivity, precision healthcare, and sustainable resource management.

Simultaneously, these integrated human-AI-robot systems will profoundly enrich cultural landscapes by fostering new forms of social interaction, knowledge sharing, artistic expression, and education. Robots and AI interfaces, capable of interpreting and responding to nuanced human emotions and cultural contexts, will catalyze innovative forms of art, entertainment, and immersive experiences that resonate deeply with human values and identities. This alignment amplifies the exchange of “cultural energy,” promoting emotional well-being, social cohesion, and cross-cultural understanding.

Thus, human-AI-robot integration stands uniquely positioned to sustainably advance both material prosperity and human flourishing by harmoniously blending economic efficiency with cultural enrichment, creating systems capable of addressing complex societal challenges with unprecedented adaptability and creativity. What is needed is a comprehensive computing infrastructure to support and usher in this integrated future.

### SUMMARY OF THE INVENTION

The inventor has conceived and reduced to practice a system and method for real-time animal training and welfare management that utilizes non-invasive brainwave and biometric monitoring, artificial intelligence, and multimodal feedback mechanisms to enhance training effectiveness, improve animal welfare, and enable structured interspecies communication.

In an embodiment, the system includes a computing device with a hardware memory and processor configured to execute software instructions that receive and process non-human animal brainwave and biometric data. The system employs a machine-learning model to analyze neural patterns, associating them with emotional states and determining a cognitive condition, which may include a training state, a welfare rating, or a predictive health condition.

In an aspect of an embodiment, the system generates an output signal indicative of the determined cognitive condition. The output may take various forms, including audio, visual, haptic, olfactory, neural stimulation, or ultrasonic reinforcement cues to facilitate real-time interaction and feedback for both human trainers and animals.

In an aspect of an embodiment, the system dynamically adjusts environmental parameters, such as temperature, humidity, ambient noise, and lighting, based on the animal’s cognitive condition. Additionally, it can generate communication alerts directed to caretakers, veterinarians, emergency responders, operational handlers, or robotic task assistants when necessary.

In an embodiment, the system modifies training sequences using reinforcement learning-based adaptation, optimizing training based on real-time biometric feedback, detected cognitive workload, and reinforcement effectiveness.

In an aspect of an embodiment, the system includes a non-invasive brainwave sensor configured to obtain and transmit brainwave data from a non-human animal. The sensor may utilize spray-on conductive polymer inks, graphene-based EEG tattoos, functional near-infrared spectroscopy (fNIRS), or a non-invasive neuromodulation interface. In another aspect, the brainwave sensor may comprise a plurality of contact sensors affixed to a cap designed for the animal’s head.

In an embodiment, the system integrates environmental sensor data with non-human animal brainwave data through a machine-learning system employing multi-modal sensor fusion techniques. These techniques combine EEG signals, biometric data, video-based pose estimation, and environmental context to refine predictive cognitive assessments.

In an aspect of an embodiment, the system incorporates a large language model (LLM) to perform AI-driven interspecies neural translation, mapping non-human EEG signals to structured embeddings cross-referenced with species-specific behavioral lexicons. The LLM further optimizes reinforcement learning-driven training sequences by dynamically adjusting behavioral conditioning techniques based on cognitive workload analysis and generating explainable AI-based insights through causal inference modeling, Bayesian decision-making frameworks, and hierarchical reinforcement learning strategies.

By integrating real-time cognitive state monitoring, adaptive training methods, and intelligent feedback mechanisms, the disclosed system significantly enhances the efficiency, ethical treatment, and scientific understanding of non-human animal training and welfare management.

### BRIEF DESCRIPTION OF THE DRAWING FIGURES

FIG. 1 shows an exemplary environment in which a system for multimodal orchestration for human-animal-robot collaborative task execution can be used, in accordance with one or more embodiments.

FIG. 2 is a block diagram illustrating components of a system for multimodal orchestration for human-animal-robot collaborative task execution, in accordance with one or more embodiments.

FIG. 3 is a block diagram illustrating details of a neural interface component, in accordance with one or more embodiments.

FIG. 4 is a block diagram illustrating details of a translation processing unit, in accordance with one or more embodiments.

FIG. 5 is a block diagram illustrating details of a multi-species output unit, in accordance with one or more embodiments.

FIG. 6 is a block diagram illustrating details of a multi-species collaboration layer, in accordance with one or more embodiments.

FIG. 7 is a block diagram illustrating details of a large language model (LLM) orchestration system, in accordance with one or more embodiments.

FIG. 8 is a block diagram illustrating details of a Simultaneous Localization and Mapping (SLAM) system, in accordance with one or more embodiments.

FIG. 9 is a block diagram illustrating an exemplary training system for multimodal orchestration for human-animal-robot collaborative task execution, in accordance with one or more embodiments.

FIG. 10 shows an exemplary environment in which a system for real-time animal training and welfare management can be used, in accordance with one or more embodiments.

FIG. 11 shows another exemplary environment in which a system for real-time animal training and welfare management can be used, in accordance with one or more embodiments.

FIG. 12 shows a block diagram of an exemplary non-invasive sensor, in accordance with one or more embodiments.

FIG. 13 is a block diagram illustrating components of a system for real-time animal training and welfare management, in accordance with one or more embodiments.

FIG. 14 shows exemplary animal brainwaves that can be analyzed using one or more embodiments.

FIG. 15 is a flow diagram illustrating an exemplary method for real-time animal training and welfare management, in accordance with one or more embodiments.

FIG. 16 is a method diagram illustrating a comprehensive process flow from data acquisition to real-time inference for bilateral human-animal communication.

FIG. 17 is a flow diagram illustrating an exemplary method for training a system for real-time animal training and welfare management, according to one or more embodiments.

FIG. 18 illustrates an exemplary computing environment on which an embodiment described herein may be implemented, in full or in part.

The drawings are not necessarily to scale. The drawings are merely schematic representations, not intended to portray specific parameters of the disclosed embodiments. The drawings are intended to depict only typical embodiments of the invention, and therefore should not be considered as limiting in scope.

### DETAILED DESCRIPTION OF THE INVENTION

The present invention provides a system and method for real-time animal training and welfare management, incorporating artificial intelligence-driven analysis of brainwave and biometric data to assess cognitive states, optimize training, and improve overall animal well-being. The disclosed system utilizes non-invasive neural and physiological sensors to capture brainwave activity, heart rate, respiration, and other biometric indicators. These signals are processed through a machine-learning system that interprets neural patterns, correlates them with emotional states, and determines cognitive conditions such as attentiveness, stress, or fatigue.

In an embodiment, the system employs a computing device configured to receive and analyze real-time brainwave and biometric data from a non-human animal. The system applies advanced machine-learning techniques to isolate neural patterns indicative of cognitive engagement, emotional state, and physiological condition. Based on these assessments, the system generates output signals that may include auditory, visual, haptic, olfactory, or neural stimulation cues, providing real-time feedback to handlers, trainers, or automated systems.

In an aspect of an embodiment, the system dynamically adjusts environmental conditions such as temperature, lighting, ambient noise, and humidity based on the detected cognitive state of the animal. This adaptive feedback mechanism ensures that training occurs under optimal conditions, reducing stress and enhancing learning efficiency. The system may further generate real-time alerts and reports for designated recipients, including veterinarians, caretakers, emergency responders, or robotic task assistants, allowing for early intervention in cases of distress or abnormal behavior.

In another embodiment, the system incorporates reinforcement learning-based adaptation techniques to modify training sequences based on real-time biometric feedback. By continuously analyzing cognitive workload and response effectiveness, the system can tailor training methods to align with an animal’s readiness and engagement levels, promoting more effective and humane training practices.

In yet another aspect of an embodiment, the system includes a non-invasive brainwave sensor that may be configured as a spray-on conductive polymer ink, a graphene-based EEG tattoo, a functional near-infrared spectroscopy sensor, or a wearable cap embedded with multiple contact sensors. These sensor configurations enable seamless acquisition of neural signals without causing discomfort or requiring invasive procedures. The brainwave data obtained from these sensors is analyzed alongside environmental sensor data to refine cognitive state assessments and enhance decision-making.

In an additional embodiment, the system integrates a large language model to facilitate interspecies neural translation, allowing non-human brainwave activity to be mapped into structured communication outputs. The system further leverages AI-based cognitive modeling to optimize behavioral conditioning and reinforcement techniques, dynamically adapting training protocols based on predictive assessments of engagement and stress levels.

By enabling a deeper understanding of animal cognitive and emotional states, the disclosed invention enhances training efficacy, promotes ethical treatment, and advances interspecies communication. The integration of machine learning, non-invasive neural sensing, and adaptive feedback mechanisms allows for intelligent and responsive training methodologies applicable to a variety of use cases, including service animal training, veterinary diagnostics, wildlife monitoring, and human-animal collaboration in complex environments.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to use in the one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

### Definitions

As used herein, “Canine” refers to members of the Canidae family, which includes domestic dogs (Canis lupus familiaris), and particularly to domestic dogs.

As used herein, “brainwave sensor” refers to a device that detects and records electrical activity in the brain, such as using electroencephalography (EEG) or similar technology. These sensors work by measuring the small voltage fluctuations generated by neural activity and translating them into readable signals.

As used herein, the term “cognitive condition,” in the context of non-human animals, refers comprehensively to their current mental and emotional state, encompassing their subjective experiences, internal perceptions, and motivational readiness. It broadly includes factors such as attentiveness, alertness, engagement, curiosity, focus, stress levels, anxiety, relaxation, boredom, fatigue, confusion, fear, excitement, and contentment, as well as motivational conditions like willingness or reluctance to participate in tasks. Additionally, cognitive condition may reflect complex emotional states derived from environmental interactions, social contexts, and training experiences. The cognitive condition can be inferred through the analysis of neural patterns derived from brain activity, behavioral indicators such as body posture, vocalizations, movements, and facial expressions, and physiological markers including but not limited to heart rate variability, cortisol or other stress-related hormone levels, respiratory patterns, temperature fluctuations, pupil dilation, and galvanic skin response. Furthermore, cognitive condition assessments may incorporate multisensory input integration, environmental context evaluation, historical behavioral trends, and predictive modeling techniques to provide a robust and nuanced understanding of the animal’s internal state. This multidimensional characterization allows the cognitive condition to reflect combined mental, emotional, and physiological states, such as simultaneously experiencing boredom and physical fatigue, nervousness coupled with high energy levels, or curiosity tempered by uncertainty, thus facilitating precise and effective animal management interventions.

### Conceptual Architecture

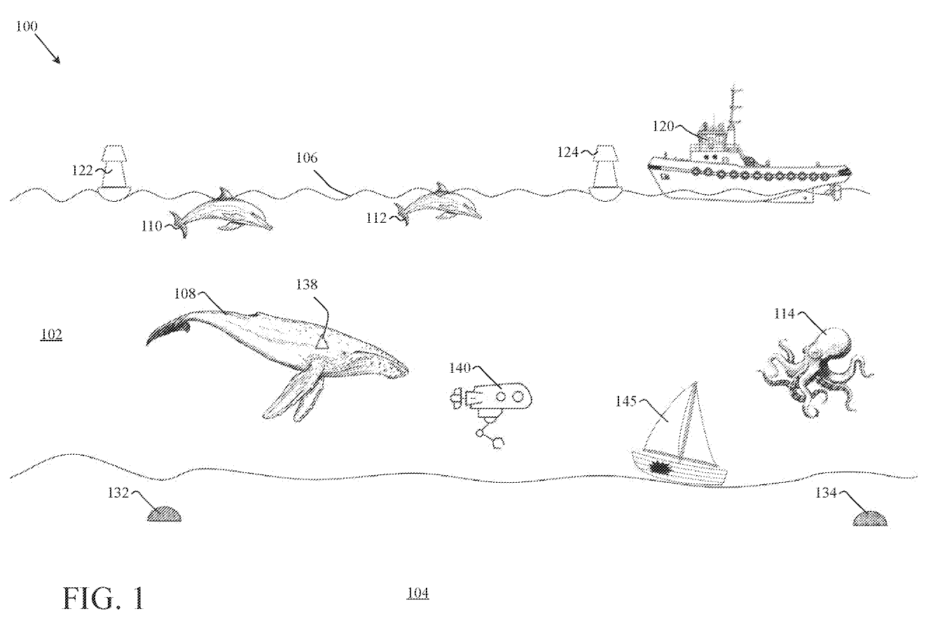

FIG. 1 shows an exemplary environment in which a system for multimodal orchestration for human-animal-robot collaborative task execution can be used, in accordance with one or more embodiments. Environment 100 can include a large body of water 102. However, disclosed embodiments are not limited to use in aquatic environments. Some embodiments may interact with land animals and/or flying animals and may be used in environments involving aquatic, land-based, and/or above-ground environments. Large body of water 102 can include an ocean (e.g., Pacific Ocean, Atlantic Ocean, etc.), a sea, (e.g., Mediterranean Sea, Baltic Sea, etc.), a lake (e.g., Lake Superior, Lake Victoria, etc.), a river (e.g., Mississippi River), a gulf (e.g., Gulf of Mexico), a bay (e.g., Hudson Bay), a strait (e.g., Bering Strait), a fjord, an estuary, a man-made reservoir, and/or other suitable large body of water.

The environment 100 can include one or more buoys, indicated as 122 and 124 in environment 100. In one or more embodiments, the buoys (122, 124) float on the surface 106 of the body of water 102, and can include a variety of equipment for sensing, receiving, storing, and/or transmitting data, as well as one or more output devices. The buoys can include one or more atmospheric sensors. The atmospheric sensors can include a wind speed sensor, wind direction sensor, air temperature sensor, air humidity sensor, barometric pressure sensor, solar radiation sensor, microphone, and/or other suitable atmospheric sensors.

The buoys can include one or more water-based sensors. The water-based sensors can include temperature sensors (to measure surface water temperature), salinity sensors (to determine the salt content of the water), pH sensors (to measure acidity/alkalinity), dissolved oxygen sensors (to monitor oxygen levels for aquatic life), and/or turbidity sensors (to measure water clarity). The water-based sensors may include wave height and direction sensors to measure ocean swell and surface conditions, current velocity sensors (to track underwater currents), tide and sea level sensors, and/or chlorophyll sensors (to estimate plankton levels and water productivity). The water-based sensors may include underwater microphones to detect underwater sounds from aquatic life and/or marine craft. The buoys can include one or more meteorological sensors, such as rain gauges and/or lightning detectors.

The buoys can include a variety of communication equipment, such as satellite transmitters (e.g., Iridium) for long-range communication. The buoys may include cellular modems, suitable for communication within areas of network coverage. The buoys may include radio transmitters to enable short-range transmission for local stations or vessels. The buoys may include Wi-Fi and/or Bluetooth modules to support local data access when in close proximity. The buoys may include GPS receivers to track buoy position and movement. Other communication systems may be present on the buoys in one or more embodiments. The buoys can include a wide variety of output devices, including, but not limited to, signal lights, audible alarms, underwater speakers, out-of-water speakers, and/or digital displays. The buoys can include a variety of computing devices, such as embedded microcontrollers, edge processors, data loggers, and/or other suitable computing equipment. In one or more embodiments, the buoys may include AI-based edge devices for advanced tasks such as detecting patterns, identifying marine life, and/or predictive analysis.

The environment 100 may further include one or more seafloor detection devices, that are located on seafloor 104, indicated at 132 and 134. Each seafloor detection device (132, 134) can include a seismometer, hydrophone array, and a wide range of other sensors and technologies for monitoring vibrations, seismic activity, and underwater sounds. The seafloor detection device can include a broadband seismometer for capturing a wide range of seismic frequencies. The seafloor detection device can further include a short-period seismometer to focus on high-frequency vibrations. The seafloor detection device can further include one or more accelerometers for measuring ground accelerations for vibrations caused by earthquakes, underwater landslides, or human-made activities such as drilling.

The hydrophone array within the seafloor detection devices can enable detecting soundwaves from marine mammals such as whales and dolphins. Other sounds may also be detected by the hydrophone array. These sounds can include sounds from underwater explosions, ship noise, or submarine movements. One or more hydrophones within the hydrophone array may be tuned for capturing low-frequency sounds from large marine animals and/or geological events. The seafloor detection devices may further include a current meter to track underwater currents that may result from tectonic and/or seismic activity.

In one or more embodiments, the seafloor detection devices may be communicatively coupled to one or more surface devices, such as buoys (122, 124), and/or ship 120. The communicative coupling can include cables, such as copper cables, fiber-optic cables, or the like, between a seafloor detection device and a buoy. The communicative coupling can include wireless communication such as RF-based communication and/or acoustic modems that can transmit data via sound waves to nearby surface buoys, ships, or other underwater devices.

The environment 100 can include ship 120. Ship 120 can contain one or more computing devices, such as a data server, virtualized computing environment, and/or other computing devices for enabling and/or supporting the multimodal orchestration system of disclosed embodiments. Additionally, ship 120 can provide long range communication to one or more remote servers, via the Internet. In one or more embodiments, ship 120 may be equipped with satellite communication (SATCOM). In embodiments, the ship 120 can be equipped with a satellite antenna. The satellite antenna can enable a connection with a geostationary satellite and/or a low-Earth-orbit (LEO) satellite, that in turn relays data to a ground station connected to the internet. The ship 120 may further be equipped with a cellular transceiver to utilize a cellular network when within the range of coastal areas.

Within the body of water 102, a wide variety of aquatic life may be present. The aquatic life can include one or more dolphins/porpoises, indicated at 110 and 112. These can include the Bottlenose Dolphin (Tursiops truncatus), Common Dolphin (Delphinus delphis), Dusky Dolphin (Lagenorhynchus obscurus), Harbor Porpoise (Phocoena phocoena), Spectacled Porpoise (Phocoena dioptrica), Orca or Killer Whale (Orcinus orca), and/or other varieties of dolphin/porpoise. These intelligent and diverse marine mammals contribute significantly to marine ecosystems and hold a special place in human culture and scientific research. Each species has its unique adaptations to its habitat, from the open ocean to coastal regions and even rivers. In embodiments, communication between the dolphins/porpoises for the multimodal orchestration system of disclosed embodiments may be accomplished by sending acoustic signals to the dolphins/porpoises and/or receiving acoustic signals from the dolphins/porpoises via buoys (122, 124) and/or seafloor detection devices (132, 134).

The aquatic life within body of water 102 can include one or more octopus/squid, indicated at 114. The octopus can include a Common Octopus (Octopus vulgaris), Giant Pacific Octopus (Enteroctopus dofleini), Mimic Octopus (Thaumoctopus mimicus), and/or other types of octopus. The squids can include a Giant Squid (Architeuthis dux), Colossal Squid (Mesonychoteuthis Hamiltoni), Common Squid (Loligo vulgaris), and/or other types of squids. Both octopuses and squids are among the most intelligent invertebrates, exhibiting behaviors that suggest advanced cognitive abilities, such as learning, problem-solving, and communication. In particular, octopuses have been known for their ability to solve complex puzzles, such as opening jars, navigating mazes, and manipulating objects in creative ways. Octopuses like the common octopus (Octopus vulgaris) have demonstrated learning through observation and trial-and-error. Additionally, some squids can learn to avoid predators by associating specific cues (like the presence of certain predators) with danger. These traits and abilities can be used for enabling multimodal orchestration for human-animal-robot collaborative task execution.

The aquatic life within body of water 102 can include one or more whales, indicated generally at 108. The whales can include Baleen whales (Mysticeti) and/or toothed whales (Odontoceti). The Baleen whales can include the Blue Whale (Balaenoptera musculus), Humpback Whale (Megaptera novaeangliae), Fin Whale (Balaenoptera physalus), Minke Whale (Balaenoptera acutorostrata), and/or other varieties of Baleen whale. The toothed whales can include a Sperm Whale (Physeter macrocephalus), Beluga Whale (Delphinapterus leucas), Narwhal (Monodon monoceros), and/or other types of toothed whale.

The environment 100 may include one or more marine life wearable electronic devices, such as indicated at 138, affixed to whale 108. The marine life wearable electronic device 138 can include one or more sensors, such as a GPS receiver. The GPS receiver may obtain geolocation data for the whale 108 at times when the whale surfaces. The marine life wearable electronic device 138 may further include one or more acoustic positioning sensors for using triangulation with other devices, such as buoys (122, 124) and/or seafloor detection devices (132, 134) to determine a relative position. The marine life wearable electronic device 138 may further include an accelerometer and gyroscope for tracking movement data, including swimming behavior, diving depth, and/or orientation.

Since whales may communicate via sound, and some whales use echolocation to navigate and find prey, the marine life wearable device 138 may include sensors to detect these sounds or transmit sounds to communicate with the whale 108. The sensors can include a hydrophone to detect vocalizations from the whale (such as songs, calls, or clicks) and record ambient underwater sounds. The marine life wearable device 138 may further include a transducer configured and disposed to emit sounds or vocalizations that can be heard by the whale, facilitating communication and/or behavioral studies. In embodiments, the transducer is tuned to output sounds in frequencies that whales can hear and interact with. In one or more embodiments, the marine life wearable device 138 may further include a haptic module. The haptic module can enable the marine life wearable device 138 to provide tactile feedback to the whale. The haptic vibrations can be delivered through components such as a waterproof vibration device that creates a physical sensation, in order to provide feedback to the whale.

The acoustic communication with whale 108 can include complex vocalizations and communication methods. These sounds can serve various purposes, including navigation, identification, mating, and social interactions. The types of sounds whales produce and the patterns they follow depend on the species, as different whales communicate in different ways. The sounds can include ‘songs.’ These songs are complex, long sequences of sounds that often repeat in patterns and can last for several minutes to hours. Male humpback whales are especially known for their songs, which may be used for mating purposes. The songs consist of different “themes” that are repeated in a specific order and may change over time. The songs can carry for miles underwater, allowing males to attract females or compete with other males.

The sounds can include clicks. The clicks can be short, sharp sounds that are used primarily for echolocation (a form of biological sonar). By emitting clicks and analyzing the returning echoes, whales can navigate and detect prey. In some species, clicks are used for communication, especially in social species like orcas and dolphins, where they may serve to coordinate group behavior or signal social intentions. The sounds can include low-frequency sounds. The low-frequency sounds vary from low-frequency moans and grunts to more intense roars, which are thought to be used for communication over long distances. The sounds are often very deep and can travel hundreds of miles across the ocean. The sounds can include non-vocal sounds, such as tail slaps. Some whales, such as humpback whales, may use physical slaps of their tails (flukes) or pectoral fins to produce sounds that may be used for communication, signaling aggression, or coordinating with others in their group. In one or more embodiments, the sound patterns, and corresponding animal behaviors may be stored in a database or other suitable format to serve as training data for one or more machine learning systems to facilitate interspecies communication between humans and/or one or more non-human animal species.

The marine life wearable device 138 further includes a power source, such as a rechargeable or replaceable battery. In some embodiments, the marine life wearable device 138 may be a disposable device with a one-time use sealed battery, such as a lithium-ion battery. In one or more embodiments, the marine life wearable device 138 may be affixed via a strap to the tail fin, or other appendage of the whale. In embodiments, the strap can be comprised of a biodegradable material that dissolves or decomposes over time, enabling the marine life wearable device 138 to fall off the whale after a period of time, such that the device does not cause any permanent discomfort for the whale. In some embodiments, the marine life wearable device 138 may be affixed via a biodegradable adhesive. The biodegradable adhesive can include a starch-based adhesive, and can be formulated to wear off after a period of time, enabling the marine life wearable device 138 to fall off the whale, such that the device does not cause any permanent discomfort for the whale.

The environment 100 may further include an autonomous underwater vehicle 140. The autonomous underwater vehicle 140 can be an electromechanical device that includes an onboard computer for receiving commands and/or data from ship 120, buoys (122, 124), marine life wearable device 138, and/or seafloor detection devices (132, 134). Embodiments can include receiving additional human communication data, and outputting the additional human communication data to one or more electromechanical devices, such as autonomous underwater vehicle 140. Embodiments can include receiving additional human communication data, and outputting the additional human communication data to one or more electronic devices, such as a remote computing device located on ship 120.

The types of tasks performed by the multimodal orchestration for human-animal-robot collaborative task execution can include search and rescue, exploration, surveillance, and/or other suitable tasks. Environment 100 can include a shipwreck 145. As an example, to determine the precise location of the shipwreck, size of the debris field of the shipwreck, and/or other information, a collaborative task involving humans (e.g., on ship 120), robots (e.g., autonomous underwater vehicle 140), and non-human animals (e.g., whale 108, octopus 114, and/or dolphins 110, 112) can be executed by using AI-enabled interspecies communication techniques, along with Simultaneous Localization and Mapping (SLAM) techniques enabled by sensors, satellite receivers, radar, lidar, RF-based triangulation, and/or other suitable techniques, as will be further described in the description for the figures that follow.

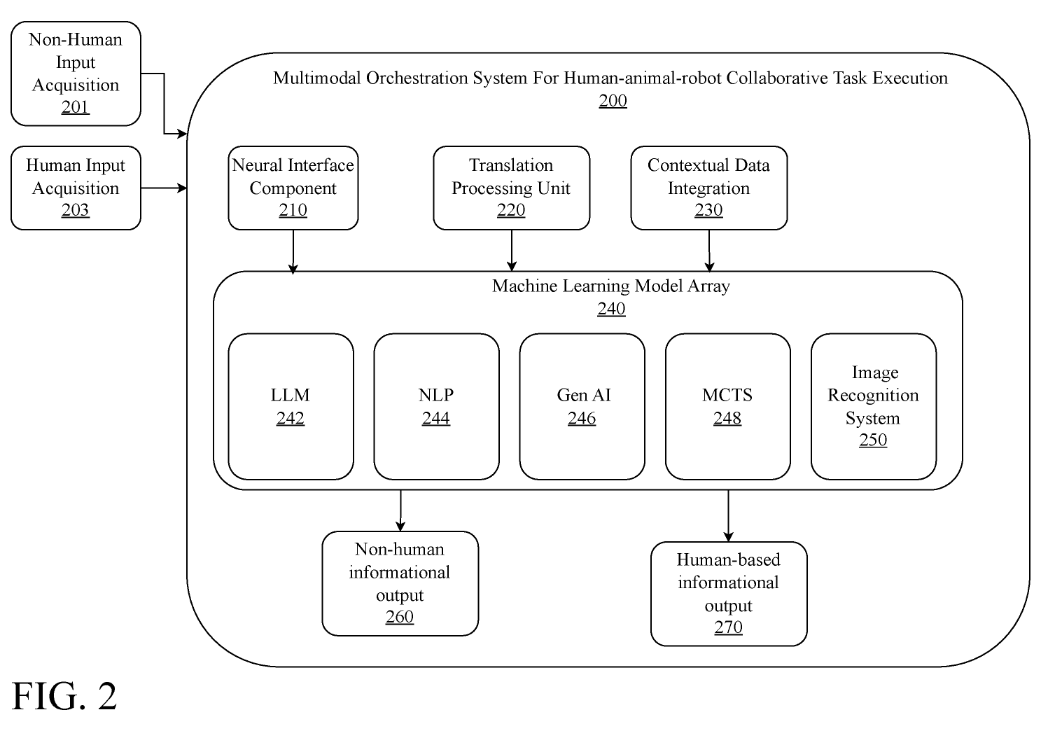

FIG. 2 is a block diagram illustrating components of a system for multimodal orchestration for human-animal-robot collaborative task execution, in accordance with one or more embodiments. System 200 can receive as input, non-human input acquisition 201, and human input acquisition 203. The non-human input acquisition 201 can include input from animals. The input can include audio input. The audio input can include vocalizations such as songs, clicks, chirps, groans, roars, and the like. The audio input can include phonemes and/or words, such as from certain species of birds that are capable of mimicking and producing phonemes from human languages. The audio input can include non-vocal sounds such as tapping or banging sounds from tapping limbs, appendages, or the like. The non-human input acquisition 201 can further include visual information such as sign language gestures, such as may be performed by various primates. The human input acquisition 203 can include spoken language, text input, sign language, and/or other suitable input. The non-human input acquisition 201 and human input acquisition 203 are input to the system for multimodal orchestration for human-animal-robot collaborative task execution 200, and the resulting output can include a non-human informational output 260, and a human-based informational output 270, thereby facilitating interspecies communication.

The system 200 can include a neural interface component 210. The neural interface component 210 can enable the detection of nuanced neural responses from animals that indicate social, emotional, and environmental interactions. The animals can include land animals, such as horses, cats, and dogs. The animals can include aquatic animals, such as whales, dolphins, fish, octopus, and squid. The animals can include birds and other flying animals. In embodiments, the neural interface component 210 may be coupled to the animals to obtain signals indicative of emotional states, and/or other communication patterns. The system 200 can include a translation processing unit 220. The translation processing unit 220 can utilize machine learning models which are trained to correlate neural patterns of animals to known behaviors, vocalizations, and intentions. The system 200 can include a contextual data integration module 230. The contextual integration module 230 can combine modalities (such as neural signals, vocalizations, gestural data, and/or scent vectors) in a multimodal fusion layer. A sliding time window provides temporal alignment, associating changes in scent concentration with concurrent neural or behavioral shifts. The outputs of the neural interface component 210, translation processing unit 220, and/or contextual data integration model 230 are input to machine learning model array 240.

Machine learning model array 240 may include one or more machine learning models, neural networks, and/or other systems for processing and interpreting input data. The machine learning model array 240 can include a large language model 242. The large language model (LLM) 242 can be trained for specific animals (e.g., species-specific or even individual-specific) and can ingest continuous streams of neural population data recorded across multiple tasks and states. These models go beyond simple language: they become multimodal encoders of animal neural signals, motor outputs, observed behaviors, and contextual cues. By structuring training data to include “high-incentive” versus “neutral” tasks, the LLM can learn when the animal’s neural signature deviates from its optimal preparatory patterns. In embodiments, the machine-learning system includes a large language model (LLM).

The machine learning model array 240 can include a natural language processing (NLP) module 244. The NLP module 244 can enable the conversion of human speech to animal-understandable patterns. The NLP module 244 can include NLP pipelines that parse human language into semantic tokens. These tokens can then be mapped onto a species-specific “neural command embedding space.” For whales, this might involve converting a request such as ‘swim to the surface’ into a neural stimulation pattern, along with an auditory output pattern such as a song or pattern of clicks. For canines, this might involve converting a request like “Fetch the red ball” into a neural stimulation pattern plus a subtle auditory or tactile cue that aligns with the dog’s pre-trained internal representations of the action “fetch” and the visual concept “red ball.”

The machine learning model array 240 can include a generative artificial intelligence (Gen AI) module 246. The Gen AI module 246 can enable supplementing training data with synthesized data, such as vocal data (e.g., canine vocalizations or whale codas), where the vocal data is created with properties such as number and regularity of signal units (clicks, barks), spectral means, and/or amplitude envelopes. The Gen AI module 246 can include a generative adversarial network (GAN), such as WaveGAN, InfoGAN, fiwGAN, and/or other suitable GAN.

The machine learning model array 240 can include a Monte Carlo Tree Search (MCTS) module 248. The MCTS module 248 can enable adaptive, look-ahead scheduling decisions. Instead of applying fixed heuristics or static load-balancing, disclosed embodiments can simulate and/or evaluate multiple future states of the pipeline before choosing the next action. By repeatedly exploring and exploiting different pipeline routing decisions (e.g., which specialist model to send partial outputs to, or how to scale certain pipeline segments), MCTS can minimize the cumulative regret over time, converging toward near-optimal scheduling policies that are robust to changing conditions, input distributions, and latency constraints. In one or more embodiments, the MCTS module 248 can enable enhanced resource allocation, such as allocating more GPUs, selecting specialized hardware accelerators, and/or adjusting batch sizes downstream.

The machine learning model array 240 can include an image recognition system 250. Image recognition system 250 may utilize machine learning to identify objects and gestures in images and video clips. The training can include obtaining a large dataset of labeled images or video clips that include the objects and/or gestures that are to be identified. Using techniques such as convolutional neural networks (CNNs), relevant features from the images are automatically extracted. A machine learning model (e.g., a deep learning model) is trained on the extracted features. Once trained, the model can be used to predict the presence of objects or gestures in new, unseen images and/or video clips. The images and/or video clips can include images of non-human animals exhibiting facial expressions, performing gestures, and/or other interpretable behaviors. Image recognition system 250 may further utilize Haar cascades for object detection. One or more embodiments can include training the Haar cascade classifier using a combination of positive samples and negative samples. The training process can include selecting the most relevant features and creating a cascade of classifiers.

The machine learning model array 240 can include, as an output, non-human informational output 260. The non-human informational output 260 can include audio output. The audio output can include species-specific audio waveforms such as clicks and songs for cetaceans, growling and/or barking sounds for canines, and so on. In an aquatic environment such as depicted in FIG. 1, the audio output may be provided by underwater speakers or other suitable transducers. The non-human informational output can include visual output, such as flashing lights, and/or patterns rendered and presented on an electronic display that is visible to the animals that are participating in the system and/or method for multimodal orchestration for human-animal-robot collaborative task execution.

The machine learning model array 240 can include, as an output, human-based informational output 270. The human-based informational output 270 can include visual information such as text and/or symbology. The human-based informational output 270 can include audio information. The audio information can include synthesized speech, tones, and/or other sounds to convey information identified by the machine learning model array 240. Referring again to the example depicted in FIG. 1, for investigation of the shipwreck 145, the non-human informational output 260 can include audio waveforms that may be interpreted by a whale 108 to swim to a location proximal to the shipwreck 145. The whale 108 may then output audio vocalizations in response to viewing the shipwreck. The output audio vocalizations can be translated by the system 200 to human-based informational output 270 for interpretations by humans. In this way, the non-human informational output 260 and the human informational output 270 can work in tandem to enable human-animal-robot collaborative task execution.

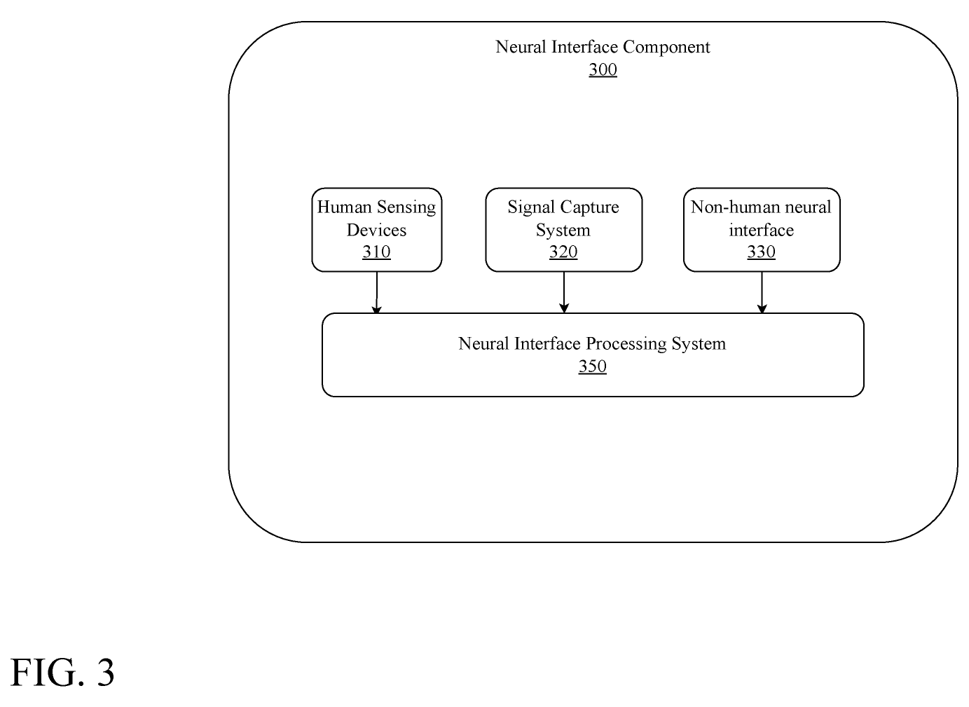

FIG. 3 is a block diagram illustrating details of a neural interface component, in accordance with one or more embodiments. Neural interface component 300 may be similar to neural interface component 210 of FIG. 2, and can include one or more human sensing devices 310. The human sensing devices 310 can include wearable sensors, such as pulse sensors, brainwave monitors, and the like. The human sensing devices 310 can further include cameras, microphones, and/or other sensors for obtaining cognitive state information from a human. The data received by the human sensing devices may be used with NLP module 244 to extract additional context and sentiment from humans participating in human-animal-robot collaborative task execution.

Neural interface component 300 can include a signal capture system 320. The signal capture system 320 can include one or more sensors for capture of neural and physiological signals without distress, adjusted for the physical characteristics of animals, such as cetaceans. In embodiments, neural sensors are embedded within wearable and/or attachable devices suited for underwater and open-ocean deployment. In embodiments, the neural interface includes components tailored to the species’ specific anatomy, such as the head or dorsal regions in whales, and made from materials that ensure durability and comfort even in the deep-sea environment.

Neural interface component 300 can include a non-human neural interface 330. The non-human neural interface 330 can include non-invasive sensors that can detect and measure animal brain activity without causing discomfort. These sensors capture neural signals associated with emotions, intentions, and responses to stimuli. The non-human neural interface 330 can further include implanted sensors. Embodiments can include surgically implanting sensor probes inside an animal’s brain. In embodiments, this technique can be used in place of a non-invasive sensor package, and can yield additional control and benefits that include the ability to record specific thoughts, evaluate mental state, and other aspects outside of the direct intent to communicate. This also enables capturing the animal’s sensory data such as vision and scent. This allows human/animals to not only communicate freely, but opens additional options for working animals. For example, a dog must pass extensive training before it can sniff drugs. With disclosed embodiments, the training may be drastically shortened by reading the animal’s brain patterns directly and identifying targeted substances via this data. As part of the training, the non-human informational output 260 (of FIG. 2) may produce a signal to cause happiness or the notion of correctness as a positive reward and drastically speed training times and animal willingness.

The output of the human sensing devices 310, signal capture system 320, and non-human neural interface 330 can be input to neural interface processing system 350. Neural interface processing system 350 can include an AI-based processing unit that analyzes and interprets vocalization data, behavioral cues, and environmental contexts to foster bidirectional communication between humans and non-human animals. Disclosed embodiments can be well-suited for applications ranging from enhancing human-animal interactions for conservation to advancing scientific understanding of animal languages, particularly in highly social and intelligent species such as sperm whales. Furthermore, disclosed embodiments can enable the use of environmental and behavioral metadata integration, as well as robust machine learning frameworks, allowing creation of an adaptable model for studying communication across various species, making disclosed embodiments adaptable to diverse animal communication needs beyond cetaceans, such as canines, primates, and other species.

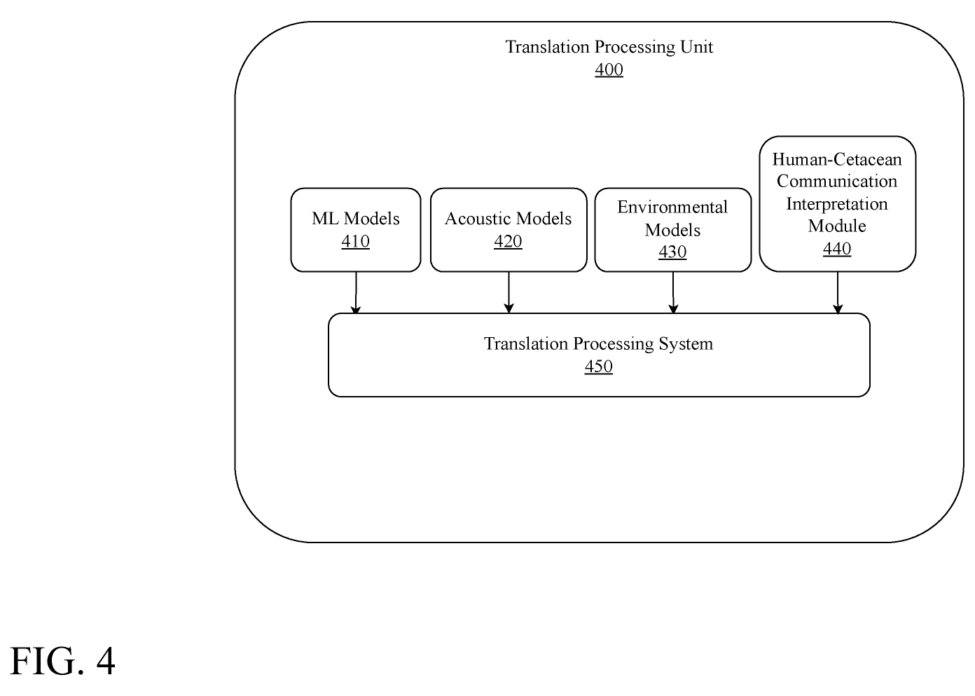

FIG. 4 is a block diagram illustrating details of a translation processing unit, in accordance with one or more embodiments. Translation processing unit 400 may be similar to translation processing unit 220 of FIG. 2, and can include one or more machine learning models 410. In embodiments, the machine learning models 410 can be trained to identify patterns in vocalization, such as codas, tempo, rhythm, ornamentation, and contextual variations like rubato. This training enables disclosed embodiments to decode structured and nuanced elements of cetacean communication, potentially revealing hierarchical or associative structures similar to human language. The translation processing unit 400 can include one or more acoustic models 420. The acoustic models 420 may enable replication of fricative production. This includes adjusting tongue position, airflow velocity, and constriction points. The translation processing unit 400 can further include one or more environmental models 430. The environmental models 430 can include probabilistic models to capture the uncertainty and variability inherent in fricative sound production and perception. The translation processing unit 400 can further include a human-cetacean communication interpretation module 440. In embodiments, the human-cetacean communication interpretation module 440 can enable mappings between humans and cetaceans. As an example, the clicks, songs, and codas of whales can be processed via machine learning, and mapped to human sentiments, such as danger, affection, joy, aggression, curiosity, and/or cooperation. In embodiments, the human sentiment can be derived from animal outputs, such as sounds made by animals, gestures made by animals, facial expressions made by animals, and so on. The danger may be represented by high-pitched whistles, rapid clicks, and/or abrupt calls. Affection may be represented by soft clicks, whistles, and/or low-pitched moans. Joy may be represented by rapid clicks, whistles, and/or varied, upbeat vocalizations. Aggression may be represented by loud, forceful vocalizations, grunts, or low-frequency rumbles. Curiosity may be represented by short bursts of clicks. Cooperation may be represented by repetitive clicking patterns. In general, cetacean sounds may be catalogued and correlated to a behavioral context.

The translation processing system 450 receives input from the ML models 410, acoustic models 420, environmental models 430, and human-cetacean communication interpretation module 440. The translation processing system 450 can be configured to convert animal neural patterns into human-comprehensible language, conveying the emotional state, intentions, and/or needs of the animal. The translation processing system 450 can further be configured to translate human speech into neural signals or cues that are meaningful and understandable to animals, allowing the animals to comprehend specific commands or sentiments directly. In embodiments, the translation processing system 450 may be configured to produce spoken translations of animal communications, thereby allowing humans to understand an animal’s emotions or needs. For instance, the system might translate a dog’s neural signals into phrases like “I am hungry” or “I’m feeling anxious.”

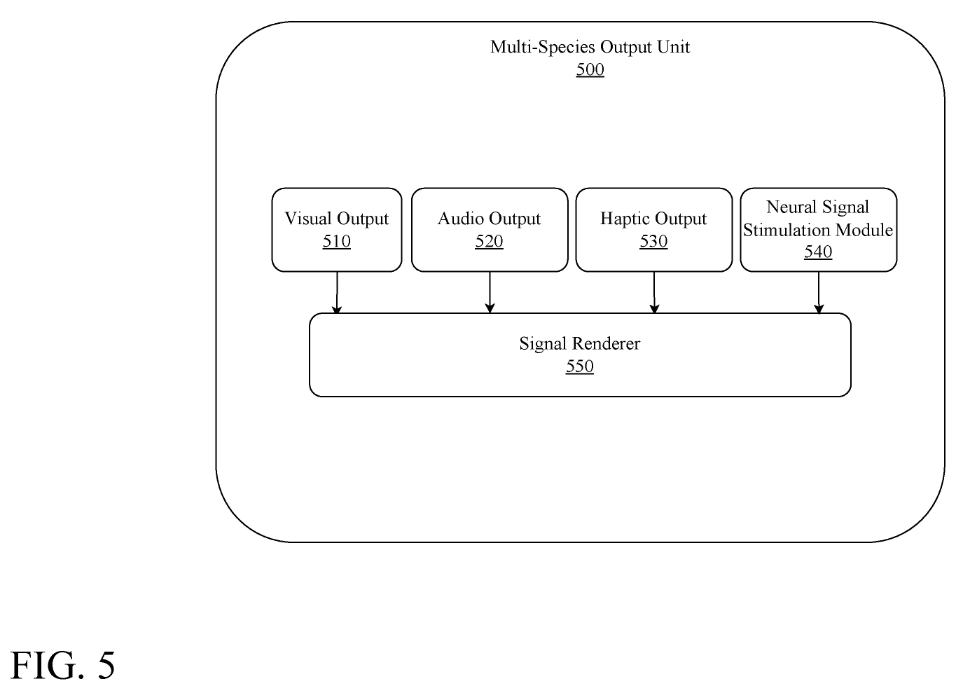

FIG. 5 is a block diagram illustrating details of a multi-species output unit 500, in accordance with one or more embodiments. The multi-species output unit is a computerized system designed to facilitate communication between humans and animals by integrating multiple sensory modalities for output. The system may generate species-specific sensory stimuli, ensuring that output signals are optimized for both human interpretation and non-human behavioral reinforcement.

The visual output subsystem 510 processes and assembles video and image data for display on an electronic screen. The visual output may be adapted to the perceptual capabilities of the target species, ensuring that animals or humans can interpret the signals effectively. In some embodiments, species-specific display methods may be used, such as color-optimized patterns for avian species or motion-based cueing for animals with reduced color vision.

The audio output subsystem 520 may be configured to generate audio waveforms that can be output through speakers, ultrasonic emitters, or bone-conduction transducers. The audio output may include species-specific vocalizations, frequencies, or tones, allowing for naturalistic reinforcement cues tailored to the target animal. In some embodiments, directional acoustic signals may be used for localized auditory training cues, preventing distraction in multi-animal environments.

The haptic output subsystem 530 may generate and modulate signals to drive vibratory actuators, pressure-based feedback devices, and electrotactile stimulation arrays. These tactile cues may be delivered via wearable sensors, embedded vests, collars, or harnesses, providing non-intrusive reinforcement signals. The system may dynamically adjust haptic intensity, duration, and frequency based on an animal’s real-time cognitive engagement state, ensuring precise and effective behavioral reinforcement.

The neural signal stimulation module 540 may include electrode-based neuromodulation devices capable of both monitoring and influencing brainwave activity. This module may be configured to record real-time neural activity and deliver low-intensity neuromodulation stimuli, influencing specific neural activation patterns to reinforce learning behaviors. In some embodiments, ultrasonic neuromodulation techniques may be integrated into the module, wherein high-frequency acoustic waves in the 20-60 kHz range stimulate cognitive engagement without direct physical contact.

The signal renderer module 550 serves as the central decision-making unit, determining the most appropriate output modality for each signal. The module may integrate signals from the visual, audio, haptic, and neural stimulation subsystems, ensuring that training reinforcement cues are species-appropriate and behaviorally effective. The multi-species output unit 500 may be integrated into, or communicatively coupled with, non-human informational output 260 and human-based informational output 270, enabling seamless coordination between training protocols, environmental feedback, and reinforcement cues.

In an embodiment of system 500, the system may include haptic output devices configured to provide real-time tactile stimuli to a non-human animal based on detected training states, emotional conditions, or cognitive engagement levels. Haptic feedback may be delivered through wearable collars, harnesses, vests, or embedded modules, employing vibration motors, pressure-actuated pads, or micro-pulsing electrodes to generate controlled stimuli that correspond to specific behavioral reinforcement patterns. The system may dynamically adjust the intensity, duration, and frequency of haptic feedback, ensuring that reinforcement cues align with the animal’s natural sensory perception and cognitive receptivity.

In some embodiments, the system may further integrate an olfactory output module, designed to dispense specific scent stimuli for reinforcing behavioral learning or mitigating stress. The olfactory module may utilize micro-controlled dispensers that release calming pheromones, food-based reward scents, or environmental enrichment aromas at optimal times during training. For example, the system may detect a state of heightened attentiveness and trigger the release of positive reinforcement scents, strengthening learned associations. Conversely, if stress or agitation indicators are detected, the system may dispense calming olfactory cues, reducing anxiety and improving receptivity to training stimuli.

The system may dynamically adjust haptic and olfactory reinforcement mechanisms based on real-time biometric analysis, ensuring that stimuli align with the animal’s cognitive state, engagement levels, and learning receptivity. The system may further be configured to adapt its reinforcement strategies based on species-specific neurophysiological and behavioral response profiles, allowing for tailored, individualized training protocols.

In an embodiment of system 500, the system may incorporate haptic and ultrasonic neuromodulation subsystems, enhancing real-time training feedback through precisely calibrated sensory interventions. The system may integrate electrotactile stimulation arrays and vibrotactile actuator networks within wearable training gear, enabling localized, spatially targeted tactile cues to reinforce conditioned responses. In some embodiments, the system may deploy adaptive impedance matching circuits, optimizing electrotactile signal strength, waveform modulation, and sensory adaptation thresholds to ensure clear and effective reinforcement without discomfort.

The system may further implement directional ultrasonic transducers, which may generate high-frequency acoustic waveforms to provide species-specific auditory reinforcement stimuli. These transducers may operate in the 20-60 kHz range, generating ultrasonic reinforcement patterns based on detected neural engagement markers. The system may dynamically modulate ultrasonic pulse sequences, tailoring auditory reinforcement based on an animal’s real-time behavioral response. In some embodiments, frequency-modulated ultrasonic waveforms may be applied to enhance attentional states, promoting adaptive cognitive engagement without requiring invasive stimulation.

The multi-species output unit 500 may be designed for integration within a wide range of training and behavioral conditioning applications, including search-and-rescue deployments, military and security working animal training, precision livestock management, and therapeutic animal interventions. The system’s multi-modal reinforcement strategies may be particularly effective in scenarios requiring cross-species coordination, non-verbal communication, and real-time cognitive state modulation.

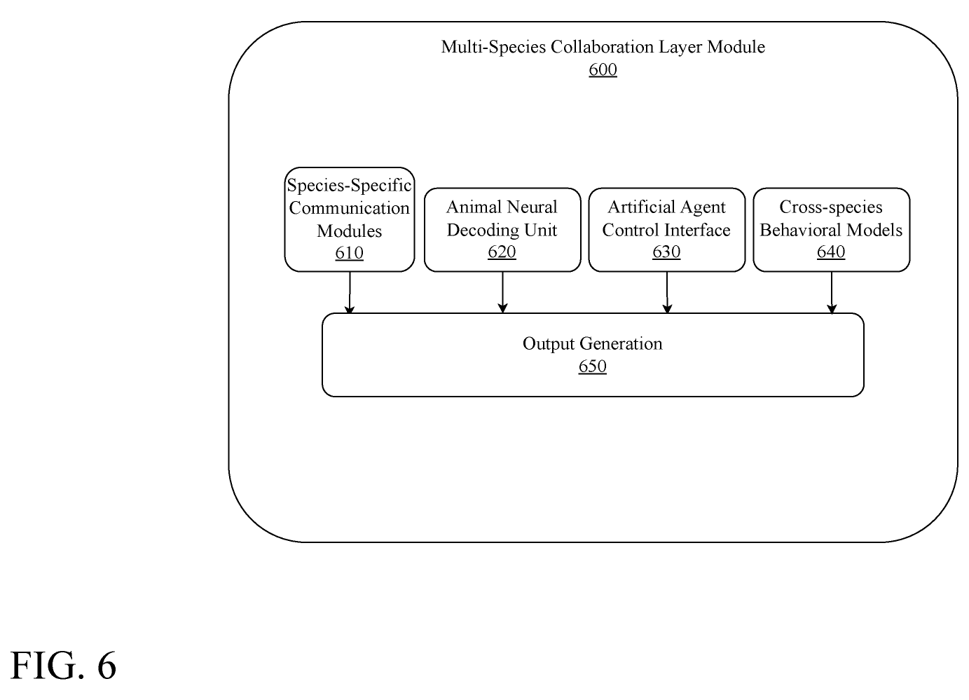

FIG. 6 is a block diagram illustrating details of a multi-species collaboration layer module 600, in accordance with one or more embodiments. The Multispecies Collaboration Layer (MCL) builds on the foundational capabilities of neural interfaces, multimodal sensory processing, and language modeling techniques, extending them to facilitate purposeful, synchronized interaction across species or between animals and machines. Its key functions include understanding interspecies vocabularies, aligning goals, and orchestrating collaborative tasks to enhance team-based efficiency between non-human animals, human handlers, and robotic assistants.

The MCL module 600 can include one or more species-specific communication modules 610, wherein each participating species, such as dogs, elephants, or avian species, may have a dedicated communication interface and representation layer tailored to their specific neural and behavioral signals. In some embodiments, the species-specific communication modules 610 may utilize contextual reinforcement learning algorithms to refine species-adaptive command-response models, optimizing how animals receive and process human or robotic instructions.

The MCL module 600 can further include an animal neural decoding unit 620, which may be configured to extract interpretable meaning vectors from an animal’s neural signals and observed behaviors. In an example implementation, a trained canine’s EEG neural activity, posture, and vocalizations may be analyzed simultaneously to generate a conceptual state embedding, representing the dog’s intentions, fatigue level, or engagement state. The animal neural decoding unit 620 may cross-validate physiological markers, such as heart rate variability and respiration rate, to improve cognitive state assessments and predictive welfare analytics.

The artificial agent control interface 630 may provide task orchestration between animals and robotic systems, wherein LIDAR, GPS, and vision-based object recognition may be used to generate abstract action representations for non-human and autonomous agents. In some embodiments, drones, robotic quadrupeds, or sensor-embedded harness systems may operate in coordination with trained animals, dynamically adjusting task difficulty, waypoint navigation, or sensory cues based on real-time behavioral feedback from the animal neural decoding unit 620.

The MCL module 600 may further include one or more cross-species behavioral models 640, which may be configured to utilize a structured library of known behavioral cues and interspecies coordination tasks. These models may assign species-specific reinforcement mappings, enabling, for example, a working dog to respond to equine herd movement cues, or a trained bird of prey to recognize robotic flight patterns in aerial tracking applications. The cross-species behavioral models 640 may dynamically optimize multi-agent task collaboration, ensuring that reinforcement learning principles apply across species-specific intelligence models.

The output generation module 650 may receive input from the species-specific communication modules 610, animal neural decoding unit 620, artificial agent control interface 630, and cross-species behavioral models 640, generating appropriate multi-modal output signals in response to detected cognitive or behavioral states. The system may produce video signals, audio commands, haptic reinforcement cues, or bioelectrical signal modulations to convey meaning and intent between human handlers, non-human animals, and autonomous agents. The output generation module 650 may process data in digital and analog formats, supporting real-time information rendering in PCM audio, raw video, compressed transmission formats, and structured reinforcement patterns tailored to species-specific responses.

In some embodiments, the Multispecies Collaboration Layer 600 may provide a unified, context-driven platform that enables animals, human handlers, and robotic systems to collaborate effectively on shared tasks. By refining interspecies neural translation dictionaries, optimizing machine learning-based intention mapping, and synchronizing cross-modal sensory feedback loops, the MCL module 600 may facilitate real-time multi-agent coordination in environments such as wildlife conservation, service animal support, search-and-rescue operations, and security task execution. In some embodiments, the MCL module 600 may be integrated with, or communicatively coupled to, system 200 of FIG. 2, ensuring interoperability with broader human-animal-robot collaborative task orchestration frameworks.

In an embodiment of system 600, the system may be configured to analyze neural, physiological, and behavioral indicators to assess an animal’s task readiness, fatigue levels, and cognitive engagement state, dynamically modifying task assignments to optimize efficiency while minimizing cognitive overload. The system may implement adaptive workload balancing mechanisms, wherein animals engaged in complex or repetitive activities may receive automated task modifications based on biometric fatigue detection and cognitive load assessments. If an animal exhibits early-stage cognitive exhaustion indicators, the system may suggest task simplification, brief rest intervals, or autonomous task redistribution to maintain optimal performance. Additionally, the system may facilitate coordination with robotic task assistants, which may assume specific workload responsibilities when an animal requires recuperation or task rotation.

The system may further support bidirectional interspecies communication, wherein animals receive real-time sensory feedback via auditory, visual, haptic, or olfactory signals, while human handlers and robotic units receive structured cognitive condition data to enhance decision-making. Applications of this system may include search-and-rescue deployments, border security operations, livestock herding, and working animal training protocols, where multi-modal communication channels improve human-animal collaboration and real-time training optimization.

In an embodiment of system 600, the system may integrate cross-species neural translation protocols, wherein AI-driven cognitive mapping frameworks may facilitate adaptive communication between non-human animals, human handlers, and robotic task assistants. The system may utilize sequence-to-sequence deep learning architectures, encoding EEG-derived cognitive states into structured neural signal embeddings, which may then be cross-referenced against species-specific behavioral lexicons to generate interpretable communication outputs.

In some embodiments, the system may implement real-time training adaptation frameworks, wherein task difficulty, reinforcement schedules, and interspecies coordination cues are dynamically adjusted based on biometric indicators of stress, attentiveness, or cognitive fatigue. The system may further incorporate hierarchical reinforcement learning techniques, allowing for progressive shaping of complex behavioral responses by leveraging transfer learning methodologies, which may enable improved training adaptability across multiple species, environmental conditions, and operational constraints.

The system may be designed for deployment in service animal training facilities, military and security operations, conservation efforts, and interspecies cognition research, where its ability to process real-time neural signals, interspecies communication cues, and machine-learning-based reinforcement strategies can enhance cross-species collaboration and optimized behavioral training strategies.

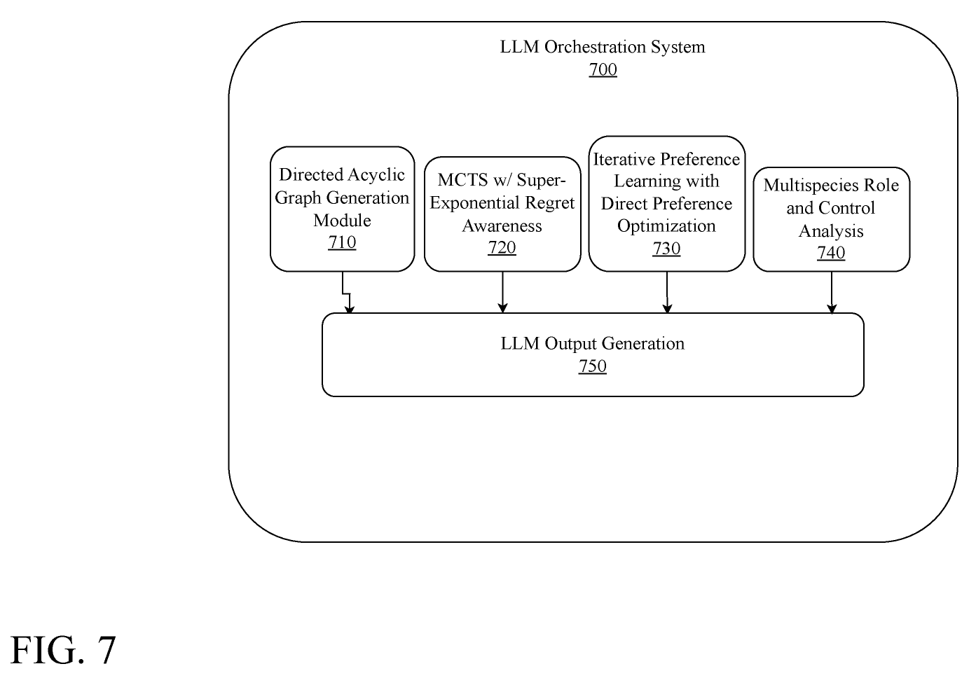

FIG. 7 is a block diagram illustrating details of a large language model (LLM) orchestration system 700, in accordance with one or more embodiments. In some embodiments, LLM orchestration system 700 may be integrated with, or communicatively coupled to, LLM 242 of FIG. 2. The system may facilitate complex reasoning, optimization, and decision-making related to multi-species task coordination, real-time training adaptation, and cognitive state classification by leveraging advanced machine learning models, structured reasoning frameworks, and reinforcement learning techniques.

LLM orchestration system 700 may include a directed acyclic graph (DAG) generation module 710, which may create a DAG representing complex workflows in which nodes correspond to reasoning steps, and edges represent transitions between partial solutions. In some embodiments, each node may encode environmental states, including animal positioning, behavior analytics, human textual instructions, and robot sensor data. DAG expansions may be performed using Monte Carlo Tree Search (MCTS)-based exploration techniques, embedding caches, semantic knowledge graphs (KGs), and preference learning algorithms. The DAG generation module 710 may be configured to represent reasoning steps required for multi-species task execution, optimizing workflow dependencies based on learned neural embeddings.

LLM orchestration system 700 may include MCTS with Super Exponential Regret Awareness module 720, which may address optimization challenges where regret—the difference between actual and optimal performance—grows at a super-exponential rate in certain AI search algorithms. In some embodiments, this module may dynamically adjust exploration-exploitation tradeoffs by fine-tuning UCT (Upper Confidence Bounds applied to Trees) parameters, mitigating excessive regret accumulation. The system may further adjust model parameters in hierarchical reinforcement learning scenarios, particularly when evaluating multi-agent interactions among human handlers, non-human animals, and robotic assistants.

The system may further incorporate an iterative preference learning module with direct preference optimization 730, which may continuously refine LLM-based decision policies. In embodiments, this module may assign preference values to node expansions within the reasoning DAG, capturing fine-grained training outcomes and behavioral response assessments. The system may collect step-level preference data by analyzing which decision paths yield more accurate animal cognitive state classifications, behavioral predictions, or environmental adaptations. Direct Preference Optimization (DPO) techniques may allow on-policy sampled data to iteratively refine LLM-based policies, ensuring that real-time cognitive state assessments become increasingly accurate over multiple training cycles.

LLM orchestration system 700 may include a multi-species role and control analysis module 740, which may model agents according to species-specific influence roles, enabling dynamic role-based reasoning optimizations. In some embodiments, the module may encourage certain agents, such as trained service animals or autonomous robotic assistants, to assume leadership roles in specific tasks, while dynamically adjusting for contextual situational factors. The module may also implement suspicious node pruning mechanisms, which may be triggered when non-human agents exhibit anomalous behaviors, such as an octopus rejecting an unhelpful object or a detection canine showing reluctance toward a particular training cue. These filtering mechanisms may enable the system to refine reasoning graphs based on contextual multi-species learning interactions.

The system may include an LLM output generation module 750, which may aggregate reasoning outputs from the directed acyclic graph generation module 710, the MCTS regret minimization module 720, the iterative preference learning module 730, and the multi-species role and control analysis module 740. The system may generate multi-modal outputs for different recipients, including human users, robotic controllers, and non-human animals. Outputs for human handlers may include structured text summaries, knowledge-based insights, and graphical dashboards providing real-time cognitive state assessments and task optimization strategies. Outputs for robotic agents may include machine-readable sensor data, real-time localization instructions, and robotic action sequences. Outputs for non-human animals may include species-specific stimuli, such as auditory reinforcement cues, vibrational haptic signals, and visual display patterns that enhance animal training and interspecies coordination.

In an embodiment of system 700, the system may employ advanced sequence modeling techniques to refine neural signature differentiation across multiple species. The system may implement contrastive learning, cross-modal embeddings, and latent space disentanglement, enabling more precise classification of emotional states such as stress, attentiveness, relaxation, and cognitive overload. The system may further incorporate explainable AI (XAI) methodologies, including decision trees, Bayesian reasoning layers, and causal graph models, to generate interpretable explanations for detected animal cognitive states. These XAI-based insights may allow human operators and robotic task assistants to make informed decisions based on AI-driven cognitive assessments.

In an embodiment, system 700 may also employ LLM-based adaptive decision-making for dynamically optimizing training sequences based on EEG and biometric data. The system may generate context-sensitive training adaptations using sequence-to-sequence deep learning models, adjusting reinforcement learning protocols based on real-time performance monitoring. The system may further integrate causal inference modeling techniques, including structural causal modeling and counterfactual reasoning, to evaluate the most effective behavioral interventions for optimizing long-term training retention, stress mitigation, and cognitive adaptation.

The system may incorporate multi-objective optimization frameworks, wherein a Pareto-optimal decision-making approach balances multiple training and welfare objectives, including maximizing learning efficiency, minimizing cognitive exhaustion, sustaining motivation, and optimizing reinforcement scheduling. In some embodiments, LLM-driven policy selection algorithms may dynamically adjust training difficulty levels, training stimuli frequency, and recovery period recommendations based on EEG-derived cognitive workload assessments.

LLM orchestration system 700 may be particularly advantageous in neuroadaptive animal training, precision veterinary diagnostics, and behavioral neuroscience research, where real-time AI-driven cognitive classifications significantly improve task adaptation, stress mitigation, and animal communication interfaces. The system may further support automated training feedback loops, wherein LLM-driven task optimizations continuously refine behavioral reinforcement strategies based on ongoing physiological monitoring.

By integrating advanced neurosymbolic reasoning, explainable AI, reinforcement learning, and large-scale decision modeling, system 700 may provide a scalable and adaptable platform for multi-species cognitive analysis, interspecies communication, and AI-driven task execution. These capabilities may be deployed in training environments, field operations, and human-animal-robot collaboration scenarios, enabling enhanced task efficiency, welfare monitoring, and cross-species intelligence modeling.

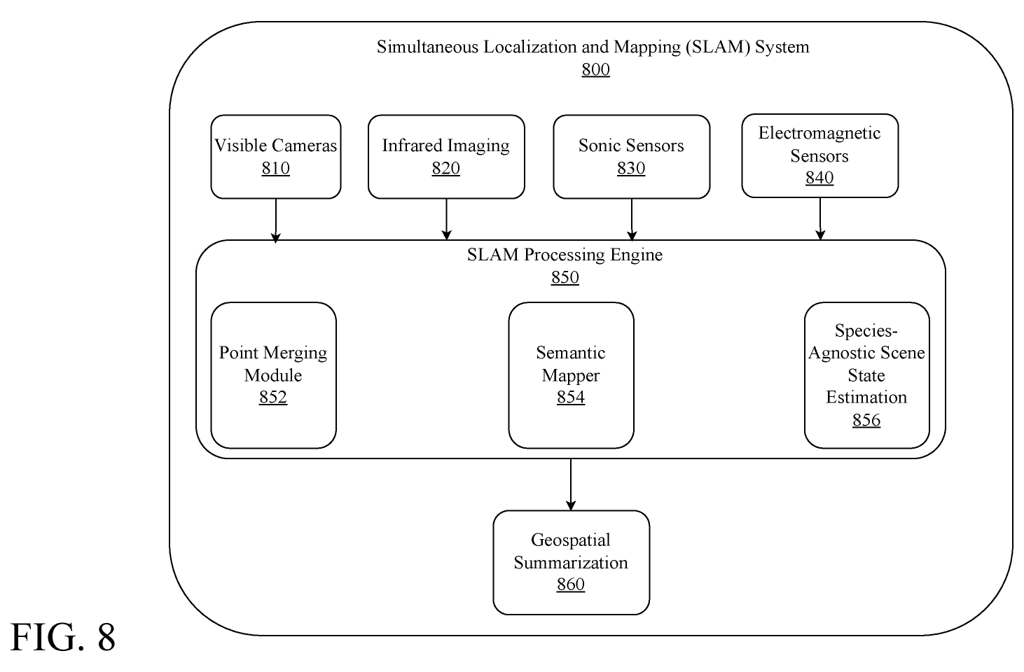

FIG. 8 is a block diagram illustrating details of a Simultaneous Localization and Mapping (SLAM) system 800, in accordance with one or more embodiments. In embodiments, SLAM system 800 may be integrated with, or communicatively coupled to, system for multimodal orchestration for human-animal-robot collaborative task execution 200 of FIG. 2. System 800 can include visible cameras 810, infrared imaging sensors 820, sonic sensors 830, and/or electromagnetic sensors 840. The visible cameras 810 may be configured to detect light in the visible spectrum (roughly 400–700 nanometers), which is the range of light perceptible to the human eye, and capture colors and details as humans see them, relying on external light sources (e.g., sunlight or artificial lighting) to illuminate a scene. The visible cameras can include wide-angle cameras, telephoto cameras, and so on. The infrared imaging sensors 820 can be configured to detect light in the infrared spectrum (beyond 700 nanometers), which is invisible to the human eye. In some embodiments, the infrared imaging sensors 820 can include thermal cameras that can capture emitted heat radiation from objects, even in complete darkness, without requiring external illumination. The sonic sensors 830 can include microphones and/or hydrophones. The microphones can include dynamic microphones, condenser microphones, electret microphones, and/or other suitable types of microphones. The hydrophones can include piezoelectric hydrophones that use piezoelectric materials to detect pressure changes in water caused by sound waves. The hydrophones can include vector sensors that measure both sound pressure and particle motion within water. The hydrophones can include a hydrophone array that includes multiple hydrophones arranged in a specific geometry to detect sound from multiple directions.

The electromagnetic sensors 840 can be configured to detect and measure electromagnetic fields or properties, such as electrical conductivity, magnetic fields, and electromagnetic radiation. The electromagnetic sensors 840 can include fluxgate magnetometers, suitable for detecting magnetic anomalies from seafloor rocks, or identifying metallic objects like shipwrecks or submarines. The electromagnetic sensors 840 can include proton precession magnetometers that can measure the magnetic field based on the precession of protons in water or a fluid. In one or more embodiments, the electromagnetic sensors can include optically pumped magnetometers, electric field detectors, capacitive sensors, electromagnetic induction sensors, and/or other suitable types of electromagnetic sensors.

The inputs from visible cameras 810, infrared imaging sensors 820, sonic sensors 830, and electromagnetic sensors 840 can be input to SLAM processing engine 850. SLAM processing engine 850 can include a point merging module 852. In embodiments, the point merging module 852 can include functions and instructions for combining multiple data points that correspond to the same real-world feature. This helps refine the map, reduce noise, and improve localization accuracy. SLAM processing engine 850 can include a semantic mapper 854. In embodiments, the semantic mapper 854 can include functions and instructions for enabling humans to interpret animal emotional states or intentions through augmented reality interfaces linked to embeddings and semantic mapping. The semantic mapper 854 may further include a Semantic Alignment Agent that can refine cross-domain mappings accordingly. Moreover, SLAM processing engine 850 can further include a species-agnostic scene state estimation module 856. In embodiments, the species-agnostic scene state estimation module 856 can include functions and instructions for utilizing data from visible light cameras to determine color and depth of a scene, enabling a 3D reconstruction of an environment. The species-agnostic scene state estimation module 856 can further include functions and instructions for utilizing data from ultraviolet (UV) and/or hyperspectral sensors, which can provide benefits as some animal signals might be visible only in UV or certain spectral bands, revealing hidden patterns (like UV-reflective markings on fish or subtle changes in an octopus’s skin). The species-agnostic scene state estimation module 856 can further include functions and instructions for utilizing data from lidar scanners that produce high-resolution point clouds for both indoor and outdoor environments. In one or more embodiments, radar can complement lidar in poor visibility conditions. The species-agnostic scene state estimation module 856 can further include functions and instructions for utilizing data from sonic and/or acoustic sensors to capture vocalizations from a wide variety of animals. Embodiments can further utilize beamforming and/or signal processing in order to locate sound sources and/or identify distress calls, barks, or other meaningful vocal patterns in non-human animals.

The output of the SLAM processing engine 850 can include a geospatial summarization 860. The geospatial summarization 860 can include data that can be rendered and presented on an electronic display to show features such as a map panel that indicates animal locations, terrain features, and environmental conditions. One or more embodiments can further include icons representing animals that are updated in real-time, displaying status indicators such as color-coded stress levels, activity patterns, and the like. The data output of geospatial summarization 860 can include data in a variety of raster, vector, and/or other suitable formats.

In an embodiment of system 800, the system may integrate edge computing infrastructures incorporating GPU acceleration, tensor processing units (TPUs), and neural network optimization modules capable of supporting computational workloads exceeding 4 teraflops, thereby enabling real-time inference while maintaining sub-150 millisecond latency constraints. Bayesian probabilistic modeling may be employed to refine uncertainty estimates, ensuring that contextual environmental variables—such as lighting variations, terrain stability, and social interactions with other animals—are incorporated into the behavioral state classification process. By optimizing real-time data processing, the system may enhance its ability to recognize cognitive states with a high degree of precision, allowing for more effective adaptation of training or intervention protocols.

The system may be designed for scalable deployment across various applications, including precision livestock management, early illness or behavioral disorder detection, collaborative human-animal robotic frameworks, and wildlife conservation monitoring. Through the integration of multi-modal behavioral analysis and real-time computational processing, the system may enable proactive cognitive and physiological assessments that enhance animal welfare, training optimization, and operational performance in diverse environments.

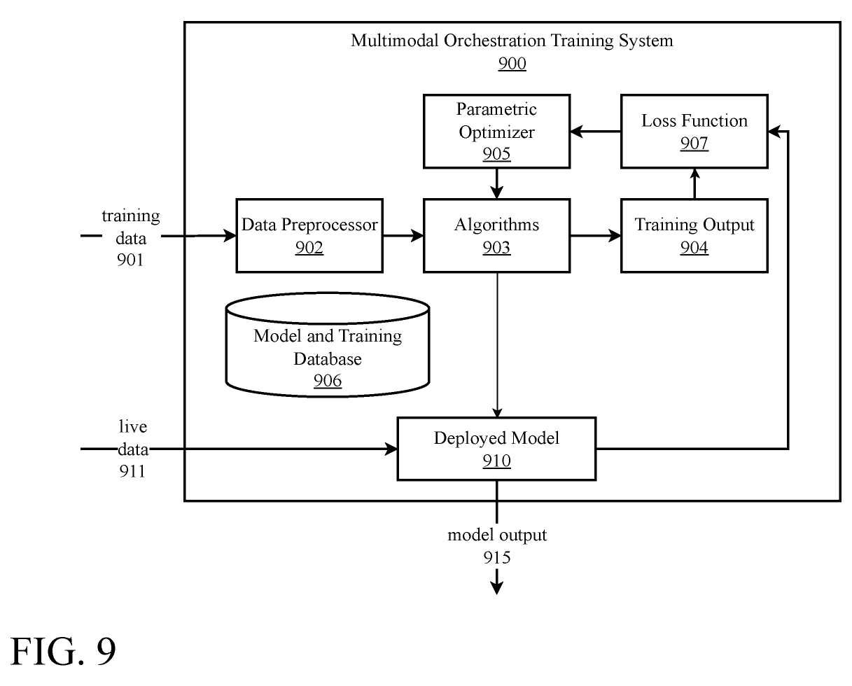

FIG. 9 is a block diagram illustrating an exemplary training system for tasks such as multimodal orchestration for human-animal-robot collaborative task execution and/or real-time animal training and welfare management, in accordance with one or more embodiments. In embodiments, system 900 may comprise a model training stage comprising a data preprocessor 902, one or more machine and/or deep learning algorithms 9 03, training output 904, a parametric optimizer 905, and a model deployment stage comprising a deployed and fully trained model 910 configured to perform tasks described herein such as enabling multimodal orchestration for human-animal-robot collaborative task execution. The system 900 may be used to train and deploy a plurality of AI subsystems in order to support the services provided by the system for multimodal orchestration for human-animal-robot collaborative task execution.

At the model training stage, a plurality of training data 901 may be received by the training system 900. Data preprocessor 902 may receive the input data (e.g., human feedback, human input data, animal input data, animal feedback, robot/sensor inputs, and the like) and perform various data preprocessing tasks on the input data to format the data for further processing. For example, data preprocessing can include, but is not limited to, tasks related to data cleansing, data deduplication, data normalization, data transformation, handling missing values, feature extraction and selection, mismatch handling, and/or the like. Data preprocessor 902 may also be configured to create a training dataset, a validation dataset, and/or a test set from the plurality of input data 901. For example, a training dataset may comprise 80% of the preprocessed input data, the validation set 10%, and the test dataset may comprise the remaining 10% of the data. The preprocessed training dataset may be fed as input into one or more machine and/or deep learning algorithms 903 to train a predictive model for tasks that can include interspecies communication, geospatial mapping, and/or object monitoring and detection.

During model training, training output 904 is produced and used to measure the accuracy and usefulness of the predictive outputs. During this process a parametric optimizer 905 may be used to perform algorithmic tuning between model training iterations. Model parameters and hyperparameters can include, but are not limited to, bias, train-test split ratio, learning rate in optimization algorithms (e.g., gradient descent), choice of optimization algorithm (e.g., gradient descent, stochastic gradient descent, of Adam optimizer, etc.), choice of activation function in a neural network layer (e.g., Sigmoid, ReLu, Tanh, etc.), the choice of cost or loss function the model will use, number of hidden layers in a neural network, number of activation unites in each layer, the drop-out rate in a neural network, number of iterations (epochs) in training the model, number of clusters in a clustering task, kernel or filter size in convolutional layers, pooling size, batch size, the coefficients (or weights) of linear or logistic regression models, cluster centroids, and/or the like. Parameters and hyperparameters may be tuned and then applied to the next round of model training. In this way, the training stage provides a machine learning training loop.

In some implementations, various accuracy metrics may be used by the training system 900 to evaluate a model’s performance. Metrics can include, but are not limited to, word error rate (WER), word information loss, cetacean response times, predicted animal response compared with actual animal response, and normalization error rate, to name a few. In one embodiment, the system may utilize a loss function 907 to measure the system’s performance. The loss function 907 compares the training outputs with an expected output and determines how the algorithm needs to be changed in order to improve the quality of the model output. During the training stage, all outputs may be passed through the loss function 907 on a continuous loop until the algorithms 903 are in a position where they can effectively be incorporated into a deployed model 915.

The test dataset can be used to test the accuracy of the model outputs. If the training model is establishing correlations that satisfy a certain criterion such as but not limited to quality of the correlations and amount of restored lost data, then it can be moved to the model deployment stage as a fully trained and deployed model 910 in a production environment making predictions based on live input data 911 (e.g., user preferences, user feedback, user inputs). Further, model correlations and restorations made by deployed model can be used as feedback and applied to model training in the training stage, wherein the model is continuously learning over time using both training data and live data and predictions. A model and training database 906 is present and configured to store training/test datasets and developed models. Database 906 may also store previous versions of models.

According to some embodiments, the one or more machine and/or deep learning models may comprise any suitable algorithm known to those with skill in the art including, but not limited to, LLMs, generative transformers, transformers, supervised learning algorithms such as: regression (e.g., linear, polynomial, logistic, etc.), decision tree, random forest, k-nearest neighbor, support vector machines, Naïve-Bayes algorithm; unsupervised learning algorithms such as clustering algorithms, hidden Markov models, singular value decomposition, and/or the like. Alternatively, or additionally, algorithms 903 may comprise a deep learning algorithm such as neural networks (e.g., recurrent, convolutional, long short-term memory networks, etc.).

In some implementations, the training system 900 automatically generates standardized model scorecards for each model produced to provide rapid insights into the model and training data, maintain model provenance, and track performance over time. These model scorecards provide insights into model framework(s) used, training data, training data specifications such as chip size, stride, data splits, baseline hyperparameters, and other factors. Model scorecards may be stored in database(s) 906.

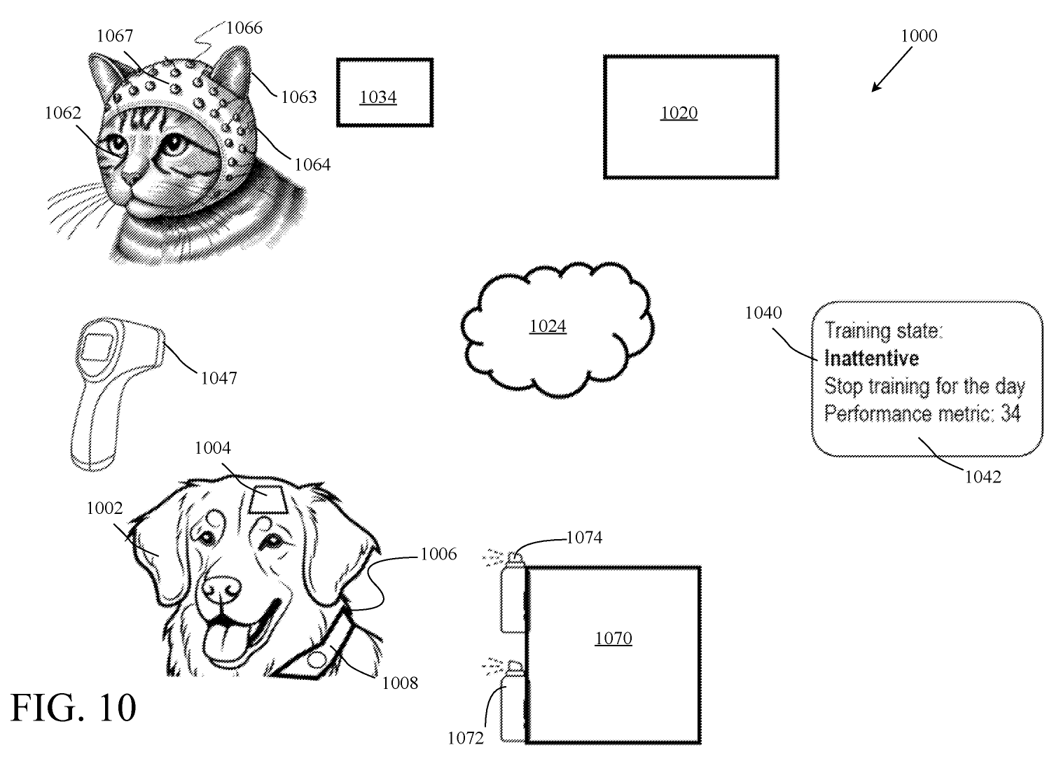

FIG. 10 shows an exemplary environment 1000 in which a system for real-time animal training and welfare management can be used, in accordance with one or more embodiments. Dog 1002 is shown, wearing a non-invasive brainwave sensor 1004. In embodiments, the non-invasive brainwave sensor 1004 may be comprised of a spray-on conductive polymer ink. In embodiments, the conductive polymer ink can include (Poly(3,4-ethylenedioxythiophene):poly(styrenesulfonate)) (PEDOT:PSS). In embodiments, the conductive polymer ink can be applied directly to the scalp or skin using a microjet printing system. In some embodiments, a small area of the dog may be shaved to expose bare skin for applying the conductive polymer ink. The ink can include additives to optimize conductivity, reduce skin impedance, and ensure mechanical durability during prolonged wear. Once applied, the ink dries into a thin, flexible film that conforms seamlessly to the skin surface, even in the presence of hair or irregular contours. In some embodiments, instead of, or in addition to, a spray-on conductive polymer ink, pre-patterned tattoo electrodes can be transferred onto the skin for neural signal acquisition. These electrodes can be composed of biocompatible materials and provide low contact impedance. In some embodiments, the epidermal tattoo sensor can include carbon nanotubes (CNTs), gold nanomaterials, and/or other suitable materials. These sensors can detect brain or muscle activity through electrical signals with high signal-to-noise ratios (SNRs), rivaling traditional invasive systems. In embodiments, the non-invasive brainwave sensor comprises a sensor comprised of conductive polymer ink. In embodiments, the conductive polymer ink comprises (Poly(3,4-ethylenedioxythiophene):poly(styrenesulfonate)) (PEDOT:PSS).

In one or more embodiments, the signals acquired by brainwave sensor 1004 are sent to a non-invasive brainwave sensor auxiliary module 1008 that may be attached to a collar 1006 worn by the dog 1002. In embodiments, the signals acquired by brainwave sensor 1004 can be sent to the brainwave sensor auxiliary module 1008 via near field communication (NFC) techniques, Bluetooth Low Energy (BLE), or other suitable techniques. One or more embodiments may utilize a custom UUID characteristic for EEG data to enable specifying the sample rate, data format, and/or other parameters for transmission of EEG data. The brainwave sensor auxiliary module 1008 can send the acquired signals to a system for real-time animal training and welfare management 1020, via network 1024. In embodiments, network 1024 can include a cellular network, WiFi network, local area network (LAN), wide area network (WAN), satellite communication network, and/or the Internet. The system for real-time animal training and welfare management 1020 can include functions and instructions to acquire brainwaves obtained by brainwave sensor 1004. The system for real-time animal training and welfare management 1020 can further include functions and instructions to perform filtering, data conditioning, and analyzing the brainwaves via machine learning models. The results of the analysis can be sent to a client device 1040 via network 1024. The client device can include a laptop computer, desktop computer, tablet computer, and/or other suitable computing device. The results produced from the system for real-time animal training and welfare management 1020 can be rendered and presented on electronic display 1042. In embodiments, the results can include a training and/or welfare rating of an animal, as determined by brainwave patterns that are analyzed by the system for real-time animal training and welfare management 1020. In embodiments, the training states that are identified can include, attentive, and inattentive, and the welfare ratings can be based on detected emotional conditions such as stressed, calm, and/or other suitable conditions. The results can further include a training recommendation. The training recommendation can include a recommendation to take a break from training, stop training for the day, continue training, and so on. Embodiments can include a non-invasive brainwave sensor, wherein the non-invasive brainwave sensor is configured and disposed to obtain brainwave data from a non-human animal, and wherein the non-invasive brainwave sensor is configured to provide the brainwave data to the computing device. In embodiments, the cognitive condition includes a training state.

The environment 1000 can further include a feline (cat) 1062. The cat 1062 can wear a sensor array 1064 to obtain brainwaves from the cat 1062. The sensor array 1064 can be implemented as a knit or crocheted headgear 1067 that the cat 1062 can wear. The headgear can include cutouts to accommodate the ears 1063 of the cat 1062. The headgear 1067 can include multiple brainwave sensors, indicated generally as 1066. The brainwaves can include signals such as EEG (Electroencephalography), EMG (Electromyography), and/or specific sensory patterns for communication or training purposes. In embodiments, the non-invasive brainwave sensor comprises a plurality of contact sensors affixed to a cap that is configured and disposed to be worn on the head of the non-human animal. The brainwaves can include different types of brainwaves (e.g., alpha, beta, delta, and/or theta waves) that can be used to analyze cognitive states. The brainwaves can be acquired and stored by data acquisition module 1034. Data acquisition module 1034 can send the brainwave data to the system for real-time animal training and welfare management 1020 via network 1024. This can enable real-time animal training and welfare management via monitoring and analyzing of animal brainwaves. As an example, the cat 1062 and or dog 1002 may generate brainwaves that are detected by sensor array 1064, and acquired by data acquisition module 1034. The data acquisition module 1034 can then send the acquired brainwave data to the system for real-time animal training and welfare management 1020 via network 1024, where the system for real-time animal training and welfare management 1020 analyzes the human brainwaves, and translates the brainwaves into a cognitive condition that can be used to derive a training state and or welfare rating for the corresponding animal. The system for real-time animal training and welfare management 1020 can derive a training state and/or a training recommendation that can be rendered and presented on a display 1042 of device 1040. Also shown on display 1042 is a performance metric. In embodiments, the performance metric can be represented as a numerical score. In some embodiments the performance metric can have a value ranging from zero to 100, with a higher value indicating better performance. In embodiments the performance metric can be calculated based on a percentage of correct training responses, a measure of animal response times as compared to nominal response times, and/or other criteria.

The environment 1000 can further include a non-contact temperature sensor 1047. The non-contact temperature sensor 1047 can include hardware and software to detect the thermal radiation emitted by an object, such as a human or animal, and converting it into a temperature reading. The non-contact temperature sensor 1047 can include a thermopile and/or pyroelectric detector that is configured to absorb the infrared energy emitted by an animal, such as dog 1002 and/or cat 1062. In one or more embodiments, the non-contact temperature sensor 1047 can be useful for detecting animal stress or discomfort by correlating changes in skin temperature patterns (e.g., a dog’s face warming in stress situations). In embodiments, environmental conditions such as temperature, humidity, ambient noise, and presence of other animals or humans, can be used to further refine a cognitive condition, welfare rating, and/or training state. Embodiments can include receiving sensor data from one or more environmental sensors; and processing the sensor data through a machine-learning system in conjunction with the non-human animal brainwave data. In embodiments, the non-contact temperature sensor 1047 can include a wireless data interface to send temperature data to data acquisition module 1034 and/or system for real-time animal training and welfare management 1020.

In embodiments, the system for real-time animal training and welfare management 1020 can interact with an animal, such as dog 1002 via a haptic and/or audio feedback the brainwave sensor auxiliary module 1008 via network 1024. The brainwave sensor auxiliary module 1008 can have one or more output devices that include speakers and/or haptic output devices such as vibrators and/or buzzers, to provide biofeedback stimulation to the dog. The dog can be trained to perform a command or action based on the biofeedback stimulation. The training can include olfactory training. The olfactory training can include a scent dispensing system 1070. The scent dispensing system 1070 can include multiple scent dispensers, indicated generally as 1072 and 1074. In embodiments the scent dispensing system can dispense scents associated with explosives, narcotics, and/or biological markers. In embodiments, the system for real-time animal training and welfare management 1020 can implement clicker training, in which a sound and/or haptic output is generated by brainwave sensor auxiliary module 1008 when the animal correctly identifies a scent, followed by a reward for the animal, reinforcing the behavior. Embodiments can include determining a training recommendation based on the training state; and rendering and presenting an audio/visual form of the training recommendation on an output device of the computing device. Embodiments can further include computing a performance metric based on the training state; and rendering and presenting an audio/visual form of the performance metric on an output device of the computing device. In embodiments, the performance metric can be a numerical score based on how efficiently, quickly, and/or accurately an animal is performing a task. In embodiments, the performance metric can be based on averages and/or standard deviation of animal reaction times, and/or other associated data.

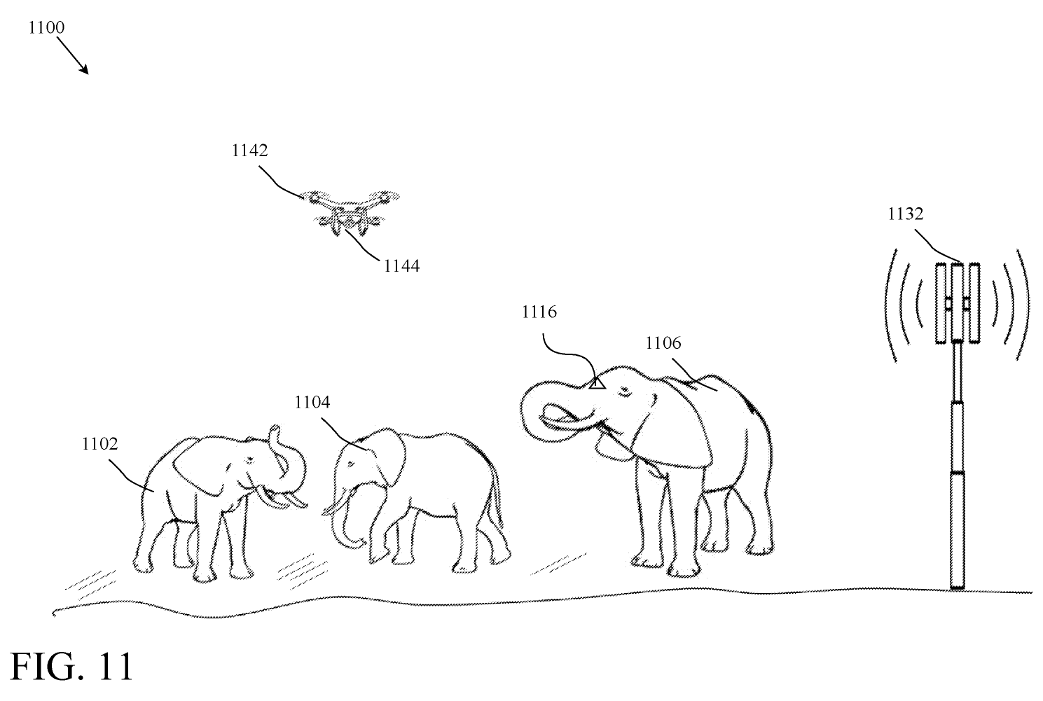

FIG. 11 shows another exemplary environment 1100 in which a system for real-time animal training and welfare management can be used, in accordance with one or more embodiments. Environment 1100 includes multiple elephants, indicated at 1102, 1104, and 1106. While elephants are shown in this example, the environment 1100 can include other animals, including wild animals, as well as livestock and domestic animals. In embodiments, a drone 1142 can operate airborne above the elephants. The drone 1142 can include a sensor array 1144 which can include one or more visible cameras, infrared cameras, hyperspectral cameras, LiDAR, and/or other sensing devices. The drone 1142 can further include a wireless data transceiver that can transmit data to radio tower 1132 to enable sending of data acquired by the drone 1142 to the system for real-time animal training and welfare management 1020 of FIG. 10. The drone 1142 can further include a speaker to output sounds that the elephants may hear, and/or output noise cancellation signals to reduce the perceived noise of the drone. The drone may further include a scent dispenser to dispense a scent that the elephants may detect. In one or more embodiments, the drone 1142 can be configured to perform a desensitization process on animals. The desensitization process can include flying multiple passes over the animals at progressively lower altitudes. In the example of FIG. 11, the desensitization process can provide the elephants with subtle cues that the drone is friendly (via calming scent patterns or gentle auditory signals), enabling the elephants to become habituated to these devices over time. The desensitization process serves to ease future collaboration in welfare monitoring tasks, such as guiding drones to areas where elephants sense poaching danger.

One or more of the elephants may further include a non-invasive biosensor, such as indicated at 1116 on elephant 1106. The non-invasive biosensor may include one or more electrodes, a power source, a signal acquisition module, a position tracker (e.g., GPS), and/or a wireless communication module. In embodiments, the non-invasive biosensor 1116 may send data to the drone 1142 and/or radio tower 1132 for upload to the system for real-time animal training and welfare management 1020 of FIG. 10. In embodiments, the non-invasive biosensor can obtain biometric data from an animal, such as heart rate, body temperature, perspiration rate, breathing rate, and so on. The biometric data can further include brainwave signals. The combination of the biometric data and the data from the sensor array 1144 of the drone 1142 may be sent to the system for real-time animal training and welfare management 1020 of FIG. 10 for analysis to determine a welfare rating. In embodiments the welfare rating can be an indicator of how stressed or calm an animal is. Thus, disclosed embodiments can provide a biometric monitoring system for wildlife that provides real-time insights into an animal’s health, behavior, and well-being. By tracking key physiological indicators such as body temperature, heart rate, and breathing rate, disclosed embodiments can significantly improve conservation efforts, animal welfare, and research. Moreover, the monitoring provided by disclosed embodiments can serve to prevent disease outbreaks by detecting fever, abnormal heart rates, or irregular breathing, allowing for early intervention before illnesses spread. Furthermore, disclosed embodiments can enable enhanced anti-poaching and security measures. Disclosed embodiments can include providing real-time alerts for unusual activity. For example, sudden spikes in stress indicators (e.g., rapid heart rate) may signal a predator threat, poaching attempt, or injury. One or more embodiments can be tailored to specific species, adjusting for differences in physiology and behavior. In embodiments, the cognitive condition includes a welfare rating.

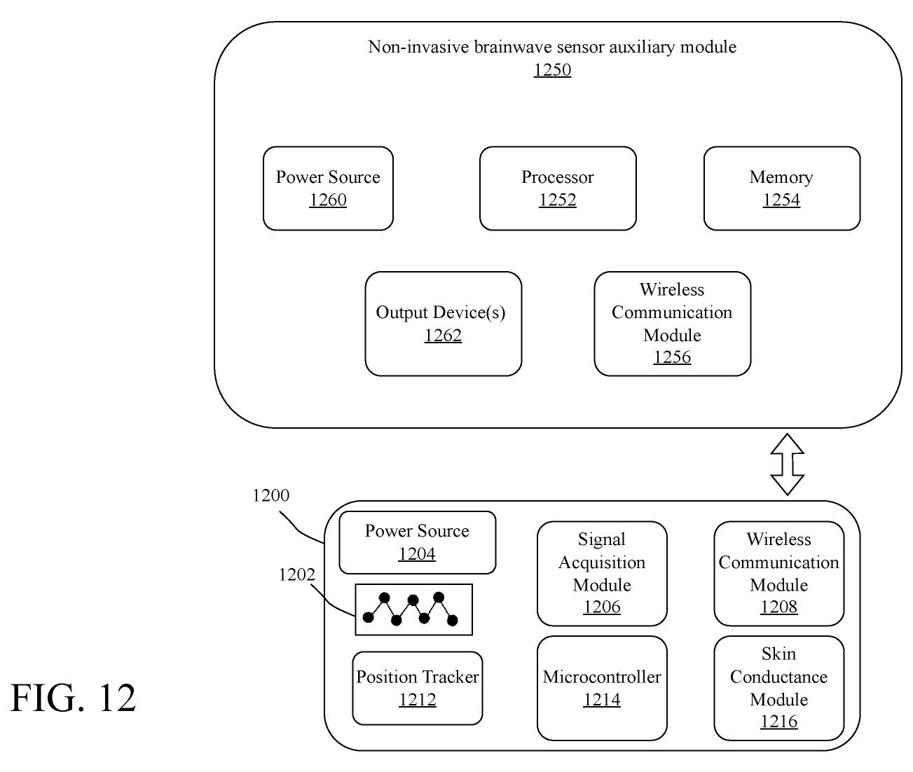

FIG. 12 shows a block diagram of an exemplary non-invasive sensor in accordance with one or more embodiments. Sensor 1200 may include a substrate 1202 that functions as an electrode layer. The substrate may comprise spray-on conductive polymer ink, such as PEDOT:PSS, and/or ultra-flexible tattoo electrodes configured to map neural signals from the animal’s head. In some embodiments, the substrate 1202 may be applied to the skin of an animal via a biodegradable adhesive. In other embodiments, a small area of the animal’s head may be shaved to expose a patch of skin, facilitating direct application of the substrate 1202. The sensor may include a flexible, biocompatible film that conforms to the skin’s surface, ensuring reliable neural signal acquisition even in the presence of fur. In some cases, an optimized spray formula may be utilized to penetrate sparse fur layers, enabling stable electrode-skin contact without requiring excessive trimming. The conductive polymer ink may further be doped with additives such as sodium chloride (NaCl) to enhance conductivity, reduce skin impedance, and improve signal acquisition quality.

Captured signals may be amplified by lightweight, on-body electronics, which may be integrated directly into the tattoo design or attached to a collar-mounted processing unit. The tattoo may be formulated for high adhesion and stretchability, ensuring it remains securely affixed despite the animal’s natural movements, including running, jumping, or exposure to moisture. In some embodiments, sensor 1200 may be deployed as a scalp-mounted sensor, such as the one depicted at 1004 in FIG. 10.

Sensor 1200 may further include a power source 1204, which may comprise a replaceable coin cell battery, a rechargeable battery, or another suitable battery type. The power source 1204 may provide energy to the signal acquisition module 1206, the wireless communication module 1208, and other components integrated within the sensor 1200. The battery may be a lithium-ion battery, though alternative power sources may be implemented based on specific use cases.

The signal acquisition module 1206 may be responsible for processing neural signals obtained from the substrate 1202. The module may include an analog-to-digital converter (ADC), which may receive a filtered input from an integrated filtering section containing low-pass filters designed to eliminate high-frequency noise. The signal acquisition module 1206 may also incorporate instrumentation amplifiers, programmable gain amplifiers, and other signal-enhancing components configured to improve the quality and resolution of the detected neural signals. In some embodiments, the signal acquisition module may further include a clock generator that provides precise timing for ADC operations. A microcontroller within the signal acquisition module 1206 may be responsible for controlling amplifier gain settings, ADC functionality, and other processing operations. The microcontroller may include an ARM Cortex processor, a RISC-V processor, or another suitable microcontroller architecture optimized for low-power, high-efficiency neural signal processing.

The wireless communication module 1208 may facilitate data transmission between sensor 1200 and external computing devices. The wireless communication module may support various communication protocols, including Near Field Communication (NFC), Bluetooth Low Energy (BLE), and Radio Frequency Identification (RFID). Additionally, the wireless communication module 1208 may include components that enable Frequency Shift Keying (FSK), Amplitude Shift Keying (ASK), and/or Phase Shift Keying (PSK) modulations for secure data transmission. In some embodiments, the wireless communication module 1208 may be equipped with long-range communication capabilities, including Wi-Fi, cellular, or satellite-based communication. These capabilities may allow the sensor 1200 to transmit neural and biometric data to the system for real-time animal training and welfare management 1020 via the internet or other suitable networks.

In some embodiments, sensor 1200 may further include a position tracker 1212, which may comprise a Global Positioning System (GPS) receiver or another suitable location-tracking system. This may allow the system to geolocate an animal’s real-time position, facilitating enhanced tracking in field environments such as wildlife monitoring, search and rescue operations, or high-performance working animal deployments.

The sensor 1200 may also include a skin conductance module 1216, which may determine electrical conductance levels of the animal’s skin based on signals received through substrate 1202. These measurements may be used to assess perspiration levels, which may serve as an indirect indicator of physiological stress. Additionally, sensor 1200 may include a microcontroller 1214, which may be coupled to the signal acquisition module 1206, position tracker 1212, and wireless communication module 1208 to manage overall sensor operations. The microcontroller may feature an ARM Cortex processor, a RISC-V processor, or another processor type optimized for real-time sensor control and data processing.

Sensor 1200 may interoperate with the non-invasive brainwave sensor auxiliary module 1250, which may serve as a collar-mounted processing unit, such as the one depicted at 1008 in FIG. 10. The non-invasive brainwave sensor auxiliary module 1250 may include a processor 1252, which may be configured to process neural and biometric data transmitted from sensor 1200. The processor 1252 may be an ARM Cortex processor, a RISC-V processor, or another processor architecture suitable for embedded computational tasks. The processor 1252 may be coupled to memory 1254, which may include a non-transitory computer-readable medium. Memory 1254 may store software instructions, calibration data, and sensor configuration settings. Memory 1254 may include random-access memory (RAM), read-only memory (ROM), Flash memory, and/or other suitable memory technologies.

The non-invasive brainwave sensor auxiliary module 1250 may include a power source 1260, which may be a replaceable battery, a rechargeable battery, or another suitable power source. Power from the power source 1260 may be distributed to components within the auxiliary module, including a wireless communication module 1256. Wireless communication module 1256 may facilitate communication between the auxiliary module and sensor 1200, using NFC, BLE, RFID, Wi-Fi, cellular, or satellite-based protocols. The wireless communication module 1256 may further support data relay functionalities, allowing sensor 1200 to offload computational tasks to an external computing device for analysis within the system for real-time animal training and welfare management 1020.

The non-invasive brainwave sensor auxiliary module 1250 may include one or more output devices 1262, which may provide sensory feedback to the animal during training. These output devices may include LED indicators, audio speakers, and haptic feedback mechanisms such as vibrators or buzzers. The LED indicators may convey operational states such as connectivity status, battery level, or alert conditions. The audio output system may generate tones, synthesized speech, or bioacoustic signals that the animal may recognize as cues for training. The haptic feedback module may impart controlled vibrational or pressure-based stimuli to reinforce behavioral responses. In some embodiments, non-invasive biosensor 1116 of FIG. 11 may be similar to sensor 1200, sharing similar design principles and integration methods.

In an embodiment of system 1200, the system may incorporate advanced non-invasive neural interface technologies, including graphene-based neural tattoos (GETs), spray-on graphene oxide conductive inks, and ultrasonic neuromodulation techniques, to enhance real-time EEG signal acquisition, cognitive monitoring, and training feedback. Graphene-based neural tattoos may be utilized as ultra-thin, biocompatible electrodes that adhere directly to the skin through van der Waals forces, eliminating the need for conductive gels or adhesive materials. These electrodes may feature hierarchical microfiber architectures, enabling deep penetration through fur layers while maintaining high signal fidelity.

In some embodiments, the system may utilize spray-on graphene oxide conductive inks, applied via aerosol jet printing, forming highly conductive, flexible, and low-impedance neural sensor arrays. These inks may integrate quantum dot-enhanced graphene composites and multi-phase nanoparticle stabilization systems, improving their durability, environmental resistance, and adaptability for long-term use.

The system may further include functional near-infrared spectroscopy (fNIRS) sensors, which may measure cortical blood flow and oxygenation levels, providing real-time insights into cognitive workload, stress responses, and neural activation patterns. Additionally, piezoelectric strain sensors and photoplethysmography (PPG) modules may be embedded in wearable collars, vests, or headgear to track heart rate variability, respiration patterns, and muscle activation levels, facilitating continuous biometric monitoring.

The system may employ tensor-based multimodal fusion architectures, synchronizing real-time neural, physiological, and environmental data to enhance cognitive state estimation. These advanced data-processing techniques may enable predictive monitoring of stress accumulation, fatigue detection, and training optimization, improving welfare-based decision-making and behavioral conditioning strategies across zoological research, veterinary diagnostics, conservation programs, and high-performance animal training applications.

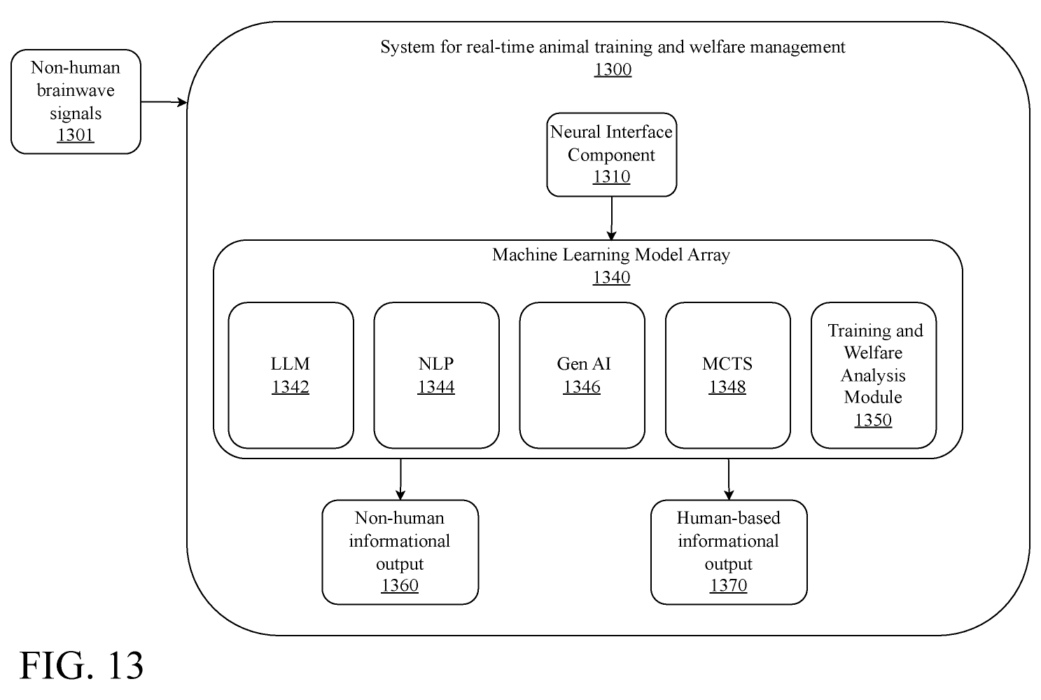

FIG. 13 is a block diagram illustrating components of a system for real-time animal training and welfare management in accordance with one or more embodiments. System 1300 may receive as input non-human brainwave signals 1301, which may include brainwave data obtained via non-invasive sensors such as spray-on conductive polymer inks, epidermal tattoo sensors, wearable headgear with integrated electrodes, and graphene-based EEG tattoos. The system may also receive physiological and biometric inputs, including electromyography (EMG), heart rate variability (HRV), respiration rate, skin conductance, body temperature, and functional near-infrared spectroscopy (fNIRS) data, enabling comprehensive real-time monitoring of an animal’s neural activity, autonomic nervous system responses, and physiological well-being. These signals may be collected from various species, including but not limited to dogs, cats, horses, oxen, primates, birds, cetaceans, and other suitable animals.

The neural interface component 1310 may enable the detection of neural responses indicative of an animal’s cognitive condition, emotional state, training state, and welfare ratings. The animals monitored may include land animals such as horses, cats, and dogs, aquatic animals such as whales, dolphins, fish, octopus, and squid, as well as birds and other flying animals. The neural interface component 1310 may be coupled to animals to obtain real-time neural signals and associated biometric data, which may then be processed within machine learning model array 1340.

The machine learning model array 1340 may include one or more machine learning models, neural networks, and other systems for processing and interpreting input data. The machine-learning model array 1340 may include a large language model (LLM) 1342, which may be trained for species-specific applications and may ingest continuous streams of neural population data recorded across multiple tasks and states. The LLM 1342 may function as a multimodal encoder, processing neural signals, motor outputs, behavioral cues, and contextual environmental factors. By structuring training data to include high-incentive versus neutral tasks, the LLM may optimize task sequencing based on an animal’s cognitive preparedness. The system may also incorporate a multi-head attention (MHA) mechanism, enabling improved self-attention by dividing inputs into multiple distinct processing heads, each attending to different aspects of the neural and behavioral data streams.

The natural language processing (NLP) module 1344 may enable the conversion of human speech into species-specific neural command patterns, facilitating real-time adaptive communication between handlers and animals. The NLP module 1344 may transform human-language training directives into interpretable stimuli for various species, such as click patterns for cetaceans, vibrational cues for canines, or visual signals for avian species.

The generative artificial intelligence (Gen AI) module 1346 may supplement training data with synthesized vocal datasets tailored for reinforcement learning applications. The Gen AI module 1346 may generate contextually accurate synthetic training signals, including vocal reinforcement tones, bioacoustic imitations, and behavioral response simulations, ensuring adaptability across diverse training environments.

The Monte Carlo Tree Search (MCTS) module 1348 may enable adaptive, look-ahead task scheduling, simulating multiple future states of the training environment before selecting optimal intervention points. The MCTS module 1348 may dynamically adjust GPU resource allocation, reinforcement learning strategies, and batch processing based on real-time cognitive workload assessments.

The training and welfare analysis module 1350 may utilize machine-learning classifiers such as Support Vector Machines (SVMs), Principal Component Analysis (PCA), Random Forests, Convolutional Neural Networks (CNNs), Recurrent Neural Networks (RNNs), and Autoencoders to interpret brainwave patterns. The module may detect anomalous neural signatures linked to epilepsy, neurological disorders, attentiveness fluctuations, stress, or fatigue, providing actionable training recommendations for improving behavioral conditioning. The training and welfare analysis module 1350 may function as an automated training companion, ensuring long-term cognitive optimization for animals engaged in high-performance tasks such as chemical detection, search and rescue, and therapeutic interventions.

System 1300 may also integrate predictive neuromodulation capabilities, allowing for anticipatory detection and mitigation of cognitive fatigue, stress, and emotional distress before overt behavioral indicators appear. The machine-learning model array 1340 may include predictive AI architectures such as Long Short-Term Memory (LSTM) networks, Kolmogorov-Arnold Networks (KANs), Kolmogorov-Arnold and Attention Networks (KaaNs), Mamba selective state space models, and Byte Latent Transformers, which may analyze EEG-derived oscillation patterns and multivariate biometric sensor data. The system may track gamma wave fluctuations for cognitive engagement, beta wave activity for attentiveness, and theta wave elevation for cognitive fatigue detection, employing hierarchical Bayesian inferencing to refine predictive accuracy.

The system may employ closed-loop neuromodulation mechanisms, wherein real-time cognitive assessments trigger targeted haptic, ultrasonic, and olfactory-based interventions via multi-species output unit 500. These interventions may include miniaturized ultrasonic transducers delivering phase-aligned acoustic waveforms, microfluidic olfactory dispensers administering controlled pheromone emissions, and wearable haptic modules generating programmable vibrational reinforcement cues. Additionally, the system may adjust environmental parameters dynamically, leveraging automated climate control systems and acoustic masking technologies to create an optimized cognitive engagement environment.

The non-human informational output 1360 may include species-specific auditory, visual, and haptic feedback mechanisms, such as bioacoustic signals for cetaceans, neural stimulation patterns for canines, and behavioral cue visualizations for handlers. The human-based informational output 1370 may provide training recommendations, welfare assessments, and cognitive state alerts, transmitting real-time performance insights to handlers, veterinarians, or automated robotic assistants.

In an aquatic environment such as depicted in FIG. 1, the audio output may be provided by underwater speakers or other suitable transducers. In the terrestrial environment such as depicted in FIG. 10, audio and/or haptic output may be provided by the brainwave sensor auxiliary module 1008 or other speakers in the environment.

In some embodiments, System 1300 may support cross-species neural translation, wherein AI-driven cognitive mapping frameworks facilitate bidirectional communication between non-human animals, humans, and robotic task assistants. The system may employ sequence-to-sequence translation architectures, converting EEG-derived cognitive states into structured feature vectors that may be cross-referenced against species-specific behavioral lexicons to enable adaptive training and interspecies collaboration.

By leveraging advanced reinforcement learning models, neurosymbolic AI, multi-modal sensor integration, and real-time adaptive training algorithms, system 1300 may provide a comprehensive platform for optimizing animal training methodologies, welfare assessments, cognitive state monitoring, and interspecies communication. Designed for use in veterinary diagnostics, conservation monitoring, advanced service animal applications, and high-performance working animal training, the system may enhance adaptive learning strategies, proactive stress management, and human-animal-robot interaction capabilities across diverse operational environments.

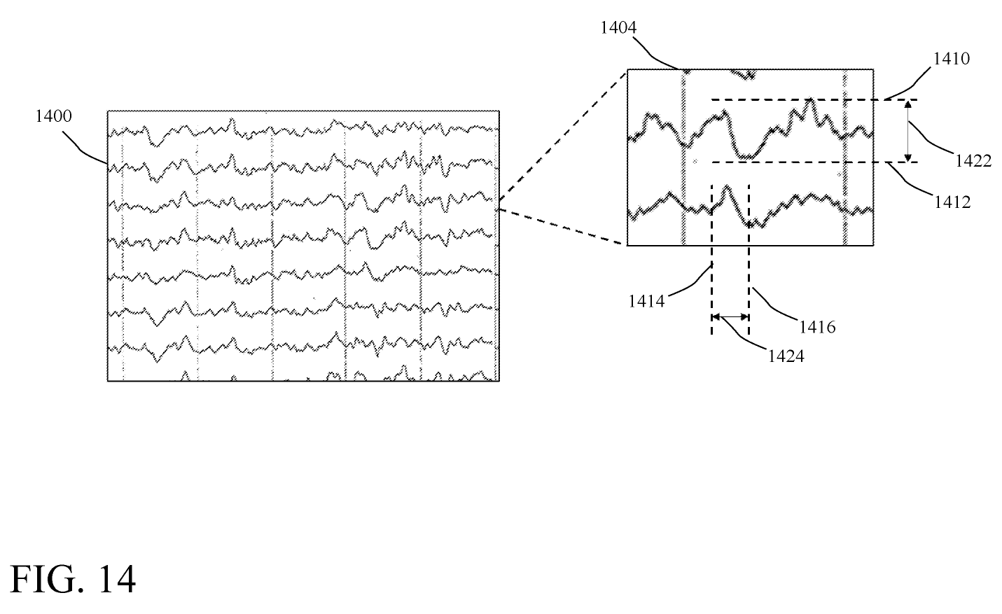

FIG. 14 shows exemplary animal brainwaves that can be analyzed using one or more embodiments. Animal brainwave signals 1400 can be obtained from a sensor such as non-invasive brainwave sensor 1004 shown in FIG. 10, or wearable sensor array 1064 as shown in FIG. 10. The brainwaves can be representative of EEG (electroencephalography) signals. Section 1404 shows additional details of the brainwaves. The brainwave signals can have a local minimum amplitude 1412 and a local maximum amplitude 1410 with a signal differential 1422. The brainwave signals can have peaks having a starting point 1414 and an ending point 1416, defining a peak duration 1424. In embodiments, the signal differential and peak duration are used as factors in determining cognitive conditions, training states, training recommendations, and/or welfare ratings.

In embodiments the animal brainwave signals 1400 can be obtained by non-invasive EEG sensors placed on an animal that can detect electrical activity. The signals shown in FIG. 14 may be preprocessed by the system 1300 for real-time animal training and welfare management of FIG. 13. The brainwave signals 1400 may be filtered to remove noise from muscle activity, blinking, and/or environmental interference. The system 1300 for real-time animal training and welfare management of FIG. 13 may further perform feature extraction on the brainwave signals 1400, in order to extract frequency-domain and/or time-domain characteristics, including delta, theta, alpha, beta, and/or gamma waves. In particular, gamma waves may have a frequency in the range of 30 Hertz to 100 Hertz, and are associated with problem-solving and perception. Deficient gamma activity has been linked to cognitive decline and learning difficulties. Accordingly, in embodiments, deficient gamma activity is indicative of a training state of inattentiveness or confusion, and can result in generating a training recommendation to stop or pause training. Similarly, beta waves, in the range of 14 Hertz to 30 Hertz, are prevalent in active thinking and problem-solving. Elevated beta wave activity can be indicative of anxiety, stress, and overthinking, while deficient beta wave activity can be indicative of lack of focus, boredom, or depression. One or more embodiments can analyze various brainwaves, such as beta waves, gamma waves, and/or other types of brainwaves, to determine a cognitive condition, training state, training recommendation, and/or welfare rating.

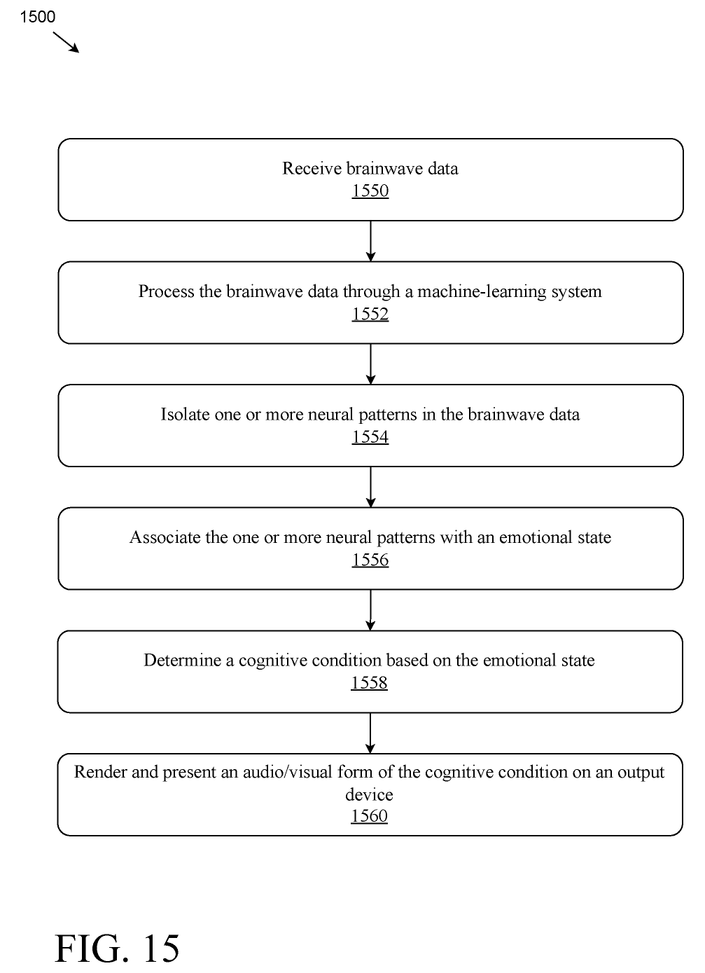

FIG. 15 is a flow diagram illustrating an exemplary method for real-time animal training and welfare management, in accordance with one or more embodiments. The method 1500 starts with receiving brainwave data at block 1550. The brainwave data can include non-human animal brainwave data. The brainwave data can be acquired from one or more non-invasive sensors. The sensors can include sensors comprised of conductive polymer ink, tattoo electrodes, and/or other suitable sensors. In embodiments, the tattoos can be placed over regions of the brain associated with emotion (e.g., limbic system regions) in order to detect neural activity indicative of emotions and/or feelings such as stress, fear, and/or happiness. In embodiments, the non-invasive brainwave sensor comprises an epidermal tattoo sensor. In embodiments, the epidermal tattoo sensor comprises carbon nanotubes (CNTs). In embodiments, the epidermal tattoo sensor comprises gold nanomaterials. One or more embodiments can include high-sensitivity brainwave capture technology that can enable non-invasive capture of brainwave patterns associated with animal responses, such as attention, excitement, or stress. This setup is especially useful in applications like service dog training, where brainwave signals can indicate readiness for commands or stress levels in various environments. The non-invasive brainwave sensor can include sensors affixed to wearable headgear, such as shown at 1067 of FIG. 10 and/or non-invasive biosensor 1116 of FIG. 11.

The method 1500 continues with processing the received brainwave data through a machine-learning system at block 1552. The machine-learning system can be trained using supervised learning techniques. Training data can include sample brainwaves obtained during known emotional states, such as fear, excitement, and/or stress. The brainwave signals can include EEG signals. The brainwave signals can be mapped to known situations, to enable creation of labeled data. The labeled data can include multiple parameters, such as observed behaviors, external stimuli, and/or physiological states. The model used can include Support Vector Machines (SVM), Random Forrest, CNNs (Convolutional Neural Networks), RNNs (Recurrent Neural Networks) / LSTMs (Long Short-Term Memory), and/or other suitable machine learning systems.

The method 1500 continues with isolating one or more neural patterns in the brainwave data at block 1554. The neural patterns can include spikes in amplitude, change in frequency, and/or other changes in characteristics of the brainwave data. The method 1500 continues with associating the one or more neural patterns with an emotional state at block 1556. In one or more embodiments, transformers and attention networks can be used for associating brainwaves with emotions and feelings once the model is trained. In embodiments, the brainwave signals can be converted into structured time-series data, by segmenting the data into time windows. Raw brainwave signals can be used to build feature maps, using techniques such as Fourier Transform, Wavelet Transform, and/or other suitable techniques. Additionally, the brainwave signals can be converted into embeddings, such as multidimensional vectors. In embodiments, the embedding can be encoded as a numerical representation of brainwave signals, mapped into a multidimensional vector space. These vectors can be used to capture the temporal, spectral, and spatial characteristics of brainwave activity, making it well-suited for machine learning models to analyze patterns. The method 1500 continues to block 1558 where a cognitive condition is determined based on the emotional state. The determination can include receiving an output from a trained machine learning model such as from machine learning model array 1340 of FIG. 13. The determination can be further based on biometric information, such as brainwave data, body temperature, heart rate, breathing rate, eyelid blinking rate, perspiration level, and/or other biometric information.

The method 1500 continues to block 1560, where an audio/visual form of the cognitive condition is rendered and presented on an output device. The output device can include a computer monitor, speakers, and/or other suitable output device. Examples of rendering and presenting an audio/visual form of an emotional state is shown at 1040 of FIG. 10, where as an example, a cognitive condition that includes a training state of “Inattentive” and a training recommendation to ‘stop training for the day’ is rendered on the electronic display 1042 of the client device 1040, along with a performance metric, indicative of the performance of the animal during the current training session. Thus, disclosed embodiments can provide an automated animal training system powered by machine learning that offers a revolutionary approach to training animals more efficiently, ethically, and effectively. By leveraging non-invasive sensors to monitor brainwaves, heart rate, and breathing rate, disclosed embodiments can analyze an animal’s cognitive condition in real time. This allows for personalized training tailored to the animal’s mental and physical state. Moreover, disclosed embodiments can detect attentiveness or fatigue and adjust training sessions accordingly, preventing wasted effort when the animal is unfocused. Recognizing when an animal is most receptive to learning ensures faster skill acquisition. Furthermore, through the use of automatic training recommendations, disclosed embodiments can increase or decrease task difficulty based on real-time cognitive feedback, making training more effective. Additionally, disclosed embodiments can help ensure training of animals is humane and ethical. By detecting signs of fatigue or mental overload, disclosed embodiments ensure animals are not pushed beyond their limits. By analyzing biometric patterns over time, disclosed embodiments can predict when an animal may struggle or excel. Furthermore, disclosed embodiments can be used with a wide variety of animals, such as dogs, horses, cats, marine mammals, and birds for training in law enforcement, therapy, and/or wildlife conservation efforts. Thus, disclosed embodiments represent a major leap forward in animal training by making the process more efficient, ethical, and personalized. By detecting cognitive conditions such as attention level, fatigue, and stress, training schedules can be optimized, thereby preventing overtraining, and enhancing learning outcomes.

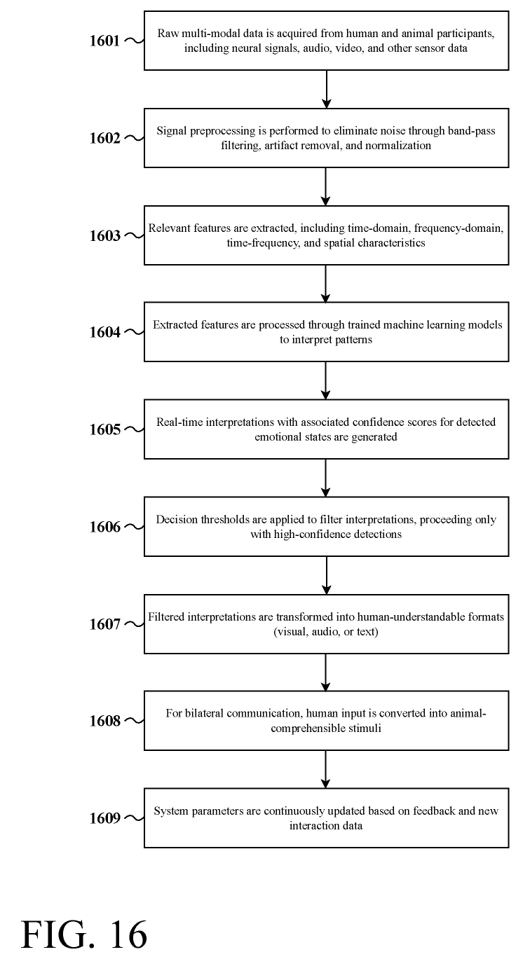

FIG. 16 is a method diagram illustrating a comprehensive process flow from data acquisition to real-time inference for bilateral human-animal communication. The system acquires raw multi-modal data from human and animal participants, collecting neural signals via neural interfaces as well as auxiliary sensor information for audio, video, or other data types, utilizing flexible conductive polymer ink or epidermal tattoo sensors applied to the animal’s skin in regions that optimize signal quality, with data acquisition components timestamping and synchronizing inputs to ensure temporal alignment of multiple data streams, potentially streaming wirelessly via Bluetooth Low Energy from the animal device to a base station 1601. The system then preprocesses these signals to clean and prepare the data for analysis, implementing procedures such as band-pass filtering to remove irrelevant frequencies, artifact removal to eliminate segments with movement artifacts or noise, normalization to adjust signal amplitude scales, and segmentation into appropriate time windows, potentially including detection of salient events such as marking points where significant changes in neural activity occur 1602. Next, the method extracts comprehensive features from the preprocessed data, deriving informative characteristics including time-domain features such as statistical measures or detection of specific neural event patterns, frequency-domain features using Fourier transforms to compute power spectral density in various frequency bands, time-frequency features applying wavelet transforms to capture transient patterns, and spatial features if multiple electrodes are used, with certain implementations employing deep feature extraction where neural networks automatically learn feature representations from raw signals 1603. The extracted features are processed through trained machine learning models that have been developed during an offline training phase where the system compiled examples linking animal neural and behavioral data to known meanings or states, with ground truth derived from labeled emotional states, controlled experiments, or unsupervised clustering, and with models potentially including deep neural networks, transformers, or hybrid systems 1604. The system generates real-time interpretations by applying the trained model to incoming features continuously, taking the current window of features and producing outputs such as emotion classifications with associated confidence scores 1605. These interpretations pass through decision thresholds or smoothing techniques to prevent spurious outputs and ensure only valid inferences proceed 1606. The system then transforms validated interpretations into human-understandable formats, presenting them as audio output such as spoken sentences, visual displays, or other appropriate modalities 1607. For bilateral implementations, the system also handles the reverse process, converting human input into forms the animal can understand, potentially through sounds or neural stimulation patterns the animal has been trained to recognize 1608. Throughout operation, the system implements supporting functions such as data storage for logging raw and processed data, error handling for managing sensor disconnections or noise issues, and multi-modal fusion for integrating different data streams, potentially requiring efficient implementation and on-device processing to maintain low latency for effective real-time operation 1609.

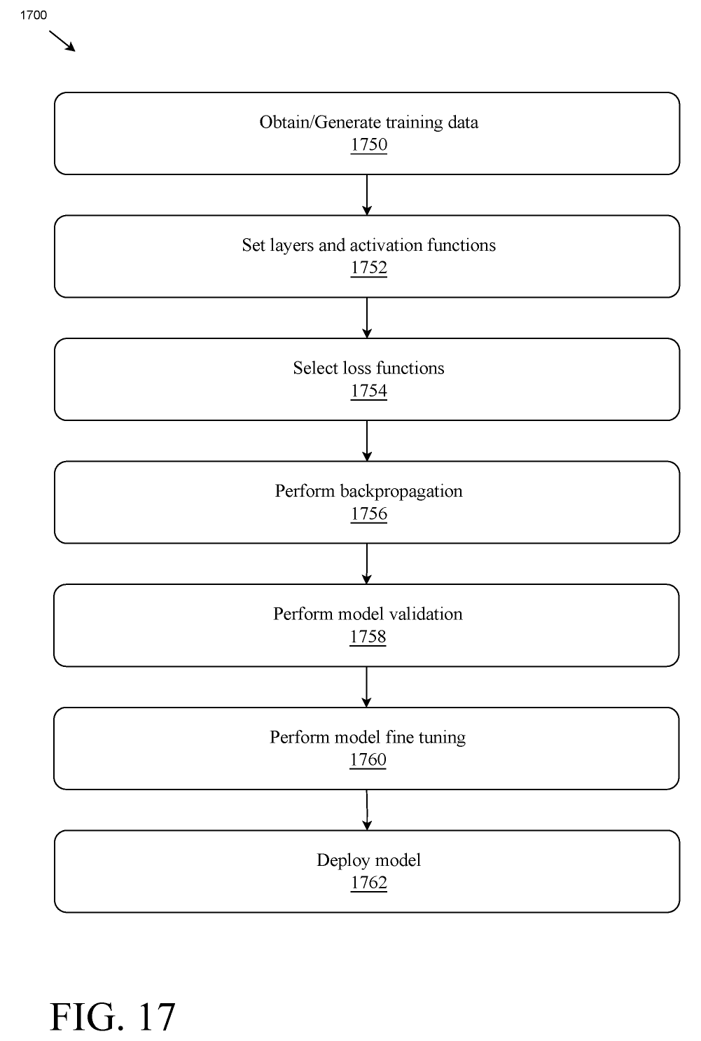

FIG. 17 is a flow diagram illustrating an exemplary method for training a system for real-time animal training and welfare management, according to one or more embodiments. The method 1700 starts with obtaining and/or generating training data at block 1750. The training data can include multiple sensor readings collected from animals in different emotional and cognitive states. The data collected can include brainwave data. The brainwave data can be categorized based on frequency ranges and corresponding associations. In embodiments, delta brainwaves (0.5–4 Hz) can be associated with deep sleep, relaxation, or unconscious states, theta brainwaves (4–8 Hz) can be associated with to relaxation, creativity, and drowsiness, alpha brainwaves (8–14 Hz) can be associated with calmness, focus, or light relaxation, beta brainwaves(14–30 Hz) can be associated with attention, alertness, and problem-solving., and gamma brainwaves (30–100 Hz) can be associated with high-level cognition, sensory perception, and learning. Additionally, the training data can include various physiological and/or biometric data. The data can include a heart rate (HR) and/or a heart rate variability (HRV). In embodiments, an elevated HR combined with a low HRV can indicate stress, excitement, or fear. In contrast, a normal HR along with an elevated high HRV can indicate a calm or relaxed state. The data can include a breathing rate. Rapid breathing can be indicative of stress, anxiety, or high alertness, while slow, rhythmic breathing can indicate a calm state. The data can further include perspiration levels. In embodiments, the perspiration levels can be associated with an emotional state. As examples, a high perspiration level can be associated with fear, stress, and/or excitement, while a low perspiration level can be associated with relaxation and/or drowsiness. In embodiments, the training data is preprocessed, such as via normalizing, filtering, and/or other techniques. Then, the data may be labeled via expert labeling, and/or other techniques.

The method 1700 continues with setting layers and activation functions at block 1752. In a neural network, layers are the building blocks that form the structure of the network. Each layer comprises a collection of neurons (also called nodes or units), and each neuron performs a specific computation on the input data. The output of one layer becomes the input to the next layer, creating a series of transformations from the input to the output. The layers can include input layers, output layers, and/or hidden layers. The activation functions introduce non-linearity into the model, allowing it to learn and represent complex patterns in the data. In embodiments, the activation functions can include a sigmoid function, a hyperbolic tangent function, a rectified linear unit (ReLU), a Leaky ReLU, softmax function, and/or other suitable activation function. The method 1700 continues to block 1754 for selecting loss functions. The loss functions are mathematical functions used in machine learning to measure the difference between the predicted values produced by the model and the actual target values from the training data. In one or more embodiments, the loss functions can include Mean Squared Error (MSE), Mean Absolute Error (MAE), Categorical Cross-Entropy, and/or other suitable loss functions. The loss functions can be used to determine if the model is sufficiently trained. The method 1700 continues to block 1756 for training the model using backpropagation. The backpropagation process can include computing gradients of the loss with respect to the weights and biases in the output layer. These gradients are propagated backward through the neural network to the hidden layer. The method 1700 continues to block 1758, where the model is validated. The validation can include using an additional set of non-human animal cognitive condition data that was not part of the original training dataset as a test dataset. In embodiments, this validation can be used to identify and correct overfitting. The method 1700 can include model fine-tuning at block 1760. The model fine-tuning can include adjusting weights and/or other hyperparameters as needed to improve model output. The method 1700 continues to block 1762, where the model is deployed for use in performing real-time animal training and welfare management.

### Exemplary Computing Environment

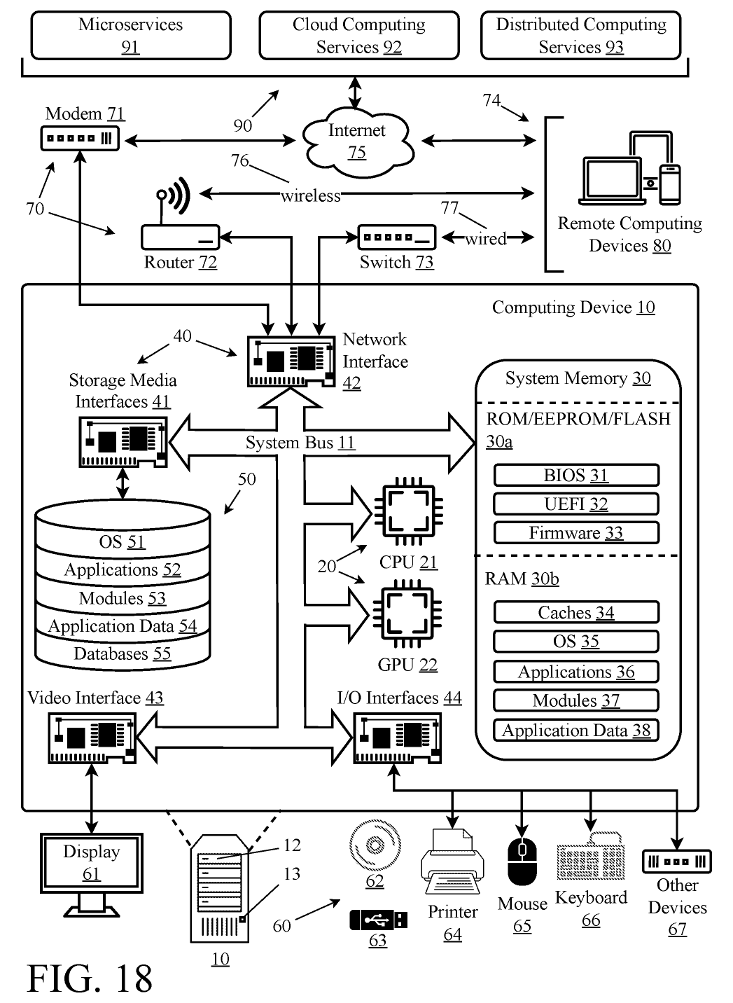

FIG. 18 illustrates an exemplary computing environment on which an embodiment described herein may be implemented, in full or in part. This exemplary computing environment describes computer-related components and processes supporting enabling disclosure of computer-implemented embodiments. Inclusion in this exemplary computing environment of well-known processes and computer components, if any, is not a suggestion or admission that any embodiment is no more than an aggregation of such processes or components. Rather, implementation of an embodiment using processes and components described in this exemplary computing environment will involve programming or configuration of such processes and components resulting in a machine specially programmed or configured for such implementation. The exemplary computing environment described herein is only one example of such an environment and other configurations of the components and processes are possible, including other relationships between and among components, and/or absence of some processes or components described. Further, the exemplary computing environment described herein is not intended to suggest any limitation as to the scope of use or functionality of any embodiment implemented, in whole or in part, on components or processes described herein.

The exemplary computing environment described herein comprises a computing device 10 (further comprising a system bus 11, one or more processors 20, a system memory 30, one or more interfaces 40, one or more non-volatile data storage devices 50), external peripherals and accessories 60, external communication devices 70, remote computing devices 80, and cloud-based services 90.

System bus 11 couples the various system components, coordinating operation of and data transmission between those various system components. System bus 11 represents one or more of any type or combination of types of wired or wireless bus structures including, but not limited to, memory busses or memory controllers, point-to-point connections, switching fabrics, peripheral busses, accelerated graphics ports, and local busses using any of a variety of bus architectures. By way of example, such architectures include, but are not limited to, Industry Standard Architecture (ISA) busses, Micro Channel Architecture (MCA) busses, Enhanced ISA (EISA) busses, Video Electronics Standards Association (VESA) local busses, a Peripheral Component Interconnects (PCI) busses also known as a Mezzanine busses, or any selection of, or combination of, such busses. Depending on the specific physical implementation, one or more of the processors 20, system memory 30 and other components of the computing device 10 can be physically co-located or integrated into a single physical component, such as on a single chip. In such a case, some or all of system bus 11 can be electrical pathways within a single chip structure.

Computing device may further comprise externally-accessible data input and storage devices 12 such as compact disc read-only memory (CD-ROM) drives, digital versatile discs (DVD), or other optical disc storage for reading and/or writing optical discs 62; magnetic cassettes, magnetic tape, magnetic disk storage, or other magnetic storage devices; or any other medium which can be used to store the desired content and which can be accessed by the computing device 10. Computing device may further comprise externally-accessible data ports or connections 12 such as serial ports, parallel ports, universal serial bus (USB) ports, and infrared ports and/or transmitter/receivers. Computing device may further comprise hardware for wireless communication with external devices such as IEEE 1394 (“Firewire”) interfaces, IEEE 802.11 wireless interfaces, BLUETOOTH® wireless interfaces, and so forth. Such ports and interfaces may be used to connect any number of external peripherals and accessories 60 such as visual displays, monitors, and touch-sensitive screens 61, USB solid state memory data storage drives (commonly known as “flash drives” or “thumb drives”) 63, printers 64, pointers and manipulators such as mice 65, keyboards 66, and other devices 67 such as joysticks and gaming pads, touchpads, additional displays and monitors, and external hard drives (whether solid state or disc-based), microphones, speakers, cameras, and optical scanners.

Processors 20 are logic circuitry capable of receiving programming instructions and processing (or executing) those instructions to perform computer operations such as retrieving data, storing data, and performing mathematical calculations. Processors 20 are not limited by the materials from which they are formed or the processing mechanisms employed therein, but are typically comprised of semiconductor materials into which many transistors are formed together into logic gates on a chip (i.e., an integrated circuit or IC). The term processor includes any device capable of receiving and processing instructions including, but not limited to, processors operating on the basis of quantum computing, optical computing, mechanical computing (e.g., using nanotechnology entities to transfer data), and so forth. Depending on configuration, computing device 10 may comprise more than one processor. For example, computing device 10 may comprise one or more central processing units (CPUs) 21, each of which itself has multiple processors or multiple processing cores, each capable of independently or semi-independently processing programming instructions. Further, computing device 10 may comprise one or more specialized processors such as a graphics processing unit (GPU) 22 configured to accelerate processing of computer graphics and images via a large array of specialized processing cores arranged in parallel. The term processor may further include: neural processing units (NPUs) or neural computing units optimized for machine learning and artificial intelligence workloads using specialized architectures and data paths; tensor processing units (TPUs) designed to efficiently perform matrix multiplication and convolution operations used heavily in neural networks and deep learning applications; application-specific integrated circuits (ASICs) implementing custom logic for domain-specific tasks; application-specific instruction set processors (ASIPs) with instruction sets tailored for particular applications; field-programmable gate arrays (FPGAs) providing reconfigurable logic fabric that can be customized for specific processing tasks; processors operating on emerging computing paradigms such as quantum computing, optical computing, mechanical computing (e.g., using nanotechnology entities to transfer data), and so forth. Depending on configuration, computing device 10 may comprise one or more of any of the above types of processors in order to efficiently handle a variety of general purpose and specialized computing tasks. The specific processor configuration may be selected based on performance, power, cost, or other design constraints relevant to the intended application of computing device 10.

System memory 30 is processor-accessible data storage in the form of volatile and/or nonvolatile memory. System memory 30 may be either or both of two types: non-volatile memory and volatile memory. Non-volatile memory 30a is not erased when power to the memory is removed, and includes memory types such as read only memory (ROM), electronically-erasable programmable memory (EEPROM), and rewritable solid-state memory (commonly known as “flash memory”). Non-volatile memory 30a is typically used for long-term storage of a basic input/output system (BIOS) 31, containing the basic instructions, typically loaded during computer startup, for transfer of information between components within computing device, or a unified extensible firmware interface (UEFI), which is a modern replacement for BIOS that supports larger hard drives, faster boot times, more security features, and provides native support for graphics and mouse cursors. Non-volatile memory 30a may also be used to store firmware comprising a complete operating system 35 and applications 36 for operating computer-controlled devices. The firmware approach is often used for purpose-specific computer-controlled devices such as appliances and Internet-of-Things (IoT) devices where processing power and data storage space is limited. Volatile memory 30b is erased when power to the memory is removed and is typically used for short-term storage of data for processing. Volatile memory 30b includes memory types such as random-access memory (RAM), and is normally the primary operating memory into which the operating system 35, applications 36, program modules 37, and application data 38 are loaded for execution by processors 20. Volatile memory 30b is generally faster than non-volatile memory 30a due to its electrical characteristics and is directly accessible to processors 20 for processing of instructions and data storage and retrieval. Volatile memory 30b may comprise one or more smaller cache memories which operate at a higher clock speed and are typically placed on the same IC as the processors to improve performance.

Interfaces 40 may include, but are not limited to, storage media interfaces 41, network interfaces 42, display interfaces 43, and input/output interfaces 44. Storage media interface 41 provides the necessary hardware interface for loading data from non-volatile data storage devices 50 into system memory 30 and storage data from system memory 30 to non-volatile data storage device 50. Network interface 42 provides the necessary hardware interface for computing device 10 to communicate with remote computing devices 80 and cloud-based services 90 via one or more external communication devices 70. Display interface 43 allows for connection of displays 61, monitors, touchscreens, and other visual input/output devices. Display interface 43 may include a graphics card for processing graphics-intensive calculations and for handling demanding display requirements. Typically, a graphics card includes a graphics processing unit (GPU) and video RAM (VRAM) to accelerate display of graphics. One or more input/output (I/O) interfaces 44 provide the necessary support for communications between computing device 10 and any external peripherals and accessories 60. For wireless communications, the necessary radio-frequency hardware and firmware may be connected to I/O interface 44 or may be integrated into I/O interface 44.

Non-volatile data storage devices 50 are typically used for long-term storage of data. Data on non-volatile data storage devices 50 is not erased when power to the non-volatile data storage devices 50 is removed. Non-volatile data storage devices 50 may be implemented using any technology for non-volatile storage of content including, but not limited to, CD-ROM drives, digital versatile discs (DVD), or other optical disc storage; magnetic cassettes, magnetic tape, magnetic disc storage, or other magnetic storage devices; solid state memory technologies such as EEPROM or flash memory; or other memory technology or any other medium which can be used to store data without requiring power to retain the data after it is written. Non-volatile data storage devices 50 may be non-removable from computing device 10 as in the case of internal hard drives, removable from computing device 10 as in the case of external USB hard drives, or a combination thereof, but computing device will typically comprise one or more internal, non-removable hard drives using either magnetic disc or solid-state memory technology. Non-volatile data storage devices 50 may store any type of data including, but not limited to, an operating system 51 for providing low-level and mid-level functionality of computing device 10, applications 52 for providing high-level functionality of computing device 10, program modules 53 such as containerized programs or applications, or other modular content or modular programming, application data 54, and databases 55 such as relational databases, non-relational databases, object oriented databases, BOSQL databases, and graph databases.

Applications (also known as computer software or software applications) are sets of programming instructions designed to perform specific tasks or provide specific functionality on a computer or other computing devices. Applications are typically written in high-level programming languages such as C++, Java, and Python, which are then either interpreted at runtime or compiled into low-level, binary, processor-executable instructions operable on processors 20. Applications may be containerized so that they can be run on any computer hardware running any known operating system. Containerization of computer software is a method of packaging and deploying applications along with their operating system dependencies into self-contained, isolated units known as containers. Containers provide a lightweight and consistent runtime environment that allows applications to run reliably across different computing environments, such as development, testing, and production systems.

The memories and non-volatile data storage devices described herein do not include communication media. Communication media are means of transmission of information such as modulated electromagnetic waves or modulated data signals configured to transmit, not store, information. By way of example, and not limitation, communication media includes wired communications such as sound signals transmitted to a speaker via a speaker wire, and wireless communications such as acoustic waves, radio frequency (RF) transmissions, infrared emissions, and other wireless media.

External communication devices 70 are devices that facilitate communications between computing device and either remote computing devices 80, or cloud-based services 90, or both. External communication devices 70 include, but are not limited to, data modems 71 which facilitate data transmission between computing device and the Internet 75 via a common carrier such as a telephone company or internet service provider (ISP), routers 72 which facilitate data transmission between computing device and other devices, and switches 73 which provide direct data communications between devices on a network. Here, modem 71 is shown connecting computing device 10 to both remote computing devices 80 and cloud-based services 90 via the Internet 75. While modem 71, router 72, and switch 73 are shown here as being connected to network interface 42, many different network configurations using external communication devices 70 are possible. Using external communication devices 70, networks may be configured as local area networks (LANs) for a single location, building, or campus, wide area networks (WANs) comprising data networks that extend over a larger geographical area, and virtual private networks (VPNs) which can be of any size but connect computers via encrypted communications over public networks such as the Internet 75. As just one exemplary network configuration, network interface 42 may be connected to switch 73 which is connected to router 72 which is connected to modem 71 which provides access for computing device 10 to the Internet 75. Further, any combination of wired 77 or wireless 76 communications between and among computing device 10, external communication devices 70, remote computing devices 80, and cloud-based services 90 may be used. Remote computing devices 80, for example, may communicate with computing device through a variety of communication channels 74 such as through switch 73 via a wired 77 connection, through router 72 via a wireless connection 76, or through modem 71 via the Internet 75. Furthermore, while not shown here, other hardware that is specifically designed for servers may be employed. For example, secure socket layer (SSL) acceleration cards can be used to offload SSL encryption computations, and transmission control protocol/internet protocol (TCP/IP) offload hardware and/or packet classifiers on network interfaces 42 may be installed and used at server devices.

In a networked environment, certain components of computing device 10 may be fully or partially implemented on remote computing devices 80 or cloud-based services 90. Data stored in non-volatile data storage device 50 may be received from, shared with, duplicated on, or offloaded to a non-volatile data storage device on one or more remote computing devices 80 or in a cloud computing service 92. Processing by processors 20 may be received from, shared with, duplicated on, or offloaded to processors of one or more remote computing devices 80 or in a distributed computing service 93. By way of example, data may reside on a cloud computing service 92, but may be usable or otherwise accessible for use by computing device 10. Also, certain processing subtasks may be sent to a microservice 91 for processing with the result being transmitted to computing device 10 for incorporation into a larger processing task. Also, while components and processes of the exemplary computing environment are illustrated herein as discrete units (e.g., OS 51 being stored on non-volatile data storage device 51 and loaded into system memory 35 for use) such processes and components may reside or be processed at various times in different components of computing device 10, remote computing devices 80, and/or cloud-based services 90.

In an implementation, the disclosed systems and methods may utilize, at least in part, containerization techniques to execute one or more processes and/or steps disclosed herein. Containerization is a lightweight and efficient virtualization technique that enables packaging and running applications and their dependencies in isolated environments called containers. One of the most popular containerization platforms is Docker, which is widely used in software development and deployment. Containerization, particularly with open-source technologies like Docker and container orchestration systems like Kubernetes, is a common approach for deploying and managing applications. Containers are created from images, which are lightweight, standalone, and executable packages that include application code, libraries, dependencies, and runtime. Images are often built from a Dockerfile or similar, which contains instructions for assembling the image. Dockerfiles are configuration files that specify how to build a Docker image. Systems like Kubernetes also support containers or CRI-O. They include commands for installing dependencies, copying files, setting environment variables, and defining runtime configurations. Docker images are stored in repositories, which can be public or private. Docker Hub is an exemplary public registry, and organizations often set up private registries for security and version control using tools such as Hub, JFrog Artifactory and Bintray, Github Packages or Container registries. Containers can communicate with each other and the external world through networking. Docker provides a bridge network by default, but can be used with custom networks. Containers within the same network can communicate using container names or IP addresses.

Remote computing devices 80 are any computing devices not part of computing device 10. Remote computing devices 80 include, but are not limited to, personal computers, server computers, thin clients, thick clients, personal digital assistants (PDAs), mobile telephones, watches, tablet computers, laptop computers, multiprocessor systems, microprocessor based systems, set-top boxes, programmable consumer electronics, video game machines, game consoles, portable or handheld gaming units, network terminals, desktop personal computers (PCs), minicomputers, main frame computers, network nodes, virtual reality or augmented reality devices and wearables, and distributed or multi-processing computing environments. While remote computing devices 80 are shown for clarity as being separate from cloud-based services 90, cloud-based services 90 are implemented on collections of networked remote computing devices 80.

Cloud-based services 90 are Internet-accessible services implemented on collections of networked remote computing devices 80. Cloud-based services are typically accessed via application programming interfaces (APIs) which are software interfaces which provide access to computing services within the cloud-based service via API calls, which are pre-defined protocols for requesting a computing service and receiving the results of that computing service. While cloud-based services may comprise any type of computer processing or storage, three common categories of cloud-based services 90 are microservices 91, cloud computing services 92, and distributed computing services 93.

Microservices 91 are collections of small, loosely coupled, and independently deployable computing services. Each microservice represents a specific computing functionality and runs as a separate process or container. Microservices promote the decomposition of complex applications into smaller, manageable services that can be developed, deployed, and scaled independently. These services communicate with each other through well-defined application programming interfaces (APIs), typically using lightweight protocols like HTTP, gRPC, or message queues such as Kafka. Microservices 91 can be combined to perform more complex processing tasks.

Cloud computing services 92 are delivery of computing resources and services over the Internet 75 from a remote location. Cloud computing services 92 provide additional computer hardware and storage on as-needed or subscription basis. Cloud computing services 92 can provide large amounts of scalable data storage, access to sophisticated software and powerful server-based processing, or entire computing infrastructures and platforms. For example, cloud computing services can provide virtualized computing resources such as virtual machines, storage, and networks, platforms for developing, running, and managing applications without the complexity of infrastructure management, and complete software applications over the Internet on a subscription basis.

Distributed computing services 93 provide large-scale processing using multiple interconnected computers or nodes to solve computational problems or perform tasks collectively. In distributed computing, the processing and storage capabilities of multiple machines are leveraged to work together as a unified system. Distributed computing services are designed to address problems that cannot be efficiently solved by a single computer or that require large-scale computational power. These services enable parallel processing, fault tolerance, and scalability by distributing tasks across multiple nodes.

Although described above as a physical device, computing device 10 can be a virtual computing device, in which case the functionality of the physical components herein described, such as processors 20, system memory 30, network interfaces 40, and other like components can be provided by computer-executable instructions. Such computer-executable instructions can execute on a single physical computing device, or can be distributed across multiple physical computing devices, including being distributed across multiple physical computing devices in a dynamic manner such that the specific, physical computing devices hosting such computer-executable instructions can dynamically change over time depending upon need and availability. In the situation where computing device 10 is a virtualized device, the underlying physical computing devices hosting such a virtualized computing device can, themselves, comprise physical components analogous to those described above, and operating in a like manner. Furthermore, virtual computing devices can be utilized in multiple layers with one virtual computing device executing within the construct of another virtual computing device. Thus, computing device 10 may be either a physical computing device or a virtualized computing device within which computer-executable instructions can be executed in a manner consistent with their execution by a physical computing device. Similarly, terms referring to physical components of the computing device, as utilized herein, mean either those physical components or virtualizations thereof performing the same or equivalent functions.

The skilled person will be aware of a range of possible modifications of the various aspects described above. Accordingly, the present invention is defined by the claims and their equivalents.

What is claimed is:

1. A computer system comprising a hardware memory, wherein the computer system is configured to execute software instructions stored on nontransitory machine-readable storage media that:
receive non-human animal brainwave data and biometric data;
process the non-human animal brainwave data and biometric data through a machine-learning system, wherein the machine-learning system is configured to:
isolate one or more neural patterns in the non-human animal brainwave data;
associate the one or more neural patterns with an emotional state of a non-human animal;
determine a cognitive condition based on the emotional state;
determine at least one of a training state, a welfare rating, or a predictive health condition based on the cognitive condition and biometric data;
generate an output signal indicative of the determined cognitive condition, wherein the output signal comprises at least one of an audio signal, a visual signal, a haptic signal, an olfactory signal, a neural stimulation signal, or an ultrasonic reinforcement cue;
adjust an environmental parameter based on the cognitive condition, wherein the environmental parameter comprises at least one of temperature, humidity, ambient noise level, lighting condition, or multimodal sensory enrichment;
generate a communication alert to a designated recipient based on the cognitive condition, wherein the designated recipient comprises at least one of a caretaker, veterinarian, emergency responder, operational handler, or robotic task assistant;
modify a training sequence using reinforcement learning-based adaptation, wherein the training sequence is adjusted based on real-time biometric feedback, detected cognitive workload, or reinforcement effectiveness;
translate non-human neural activity into structured communication outputs, wherein the structured communication outputs include neural embeddings mapped to an interspecies translation model; and
render and present an auditory or visual form of at least one of the cognitive condition, training state, welfare rating, predictive health condition, environmental adjustment, training adaptation, or interspecies communication output on an output device of the computer system.

2. The computer system of claim 1, wherein the cognitive condition includes a training state.

3. The computer system of claim 2, further comprising programming instructions that, when executed by the computer system, cause the computer system to:
determine a training recommendation based on the training state, wherein the training recommendation is adapted using reinforcement learning algorithms based on real-time biometric and cognitive workload feedback; and
render and present an audio/visual form of the training recommendation on an output device of the computing device.

4. The computer system of claim 2, further comprising programming instructions that, when executed by the computer system, cause the computer system to:
compute a performance metric based on the training state, wherein the performance metric is derived using AI-driven cognitive state modeling, behavioral tracking, and predictive workload estimation; and
render and present an audio/visual form of the performance metric on an output device of the computing device.

5. The computer system of claim 1, wherein the cognitive condition includes a welfare rating.

6. The computer system of claim 1, further comprising a non-invasive brainwave sensor, wherein the non-invasive brainwave sensor is configured to obtain brainwave data from a non-human animal, and wherein the non-invasive brainwave sensor is configured to provide the brainwave data to the computer system, wherein the non-invasive brainwave sensor comprises at least one of spray-on conductive polymer inks, graphene-based EEG tattoos, functional near-infrared spectroscopy (fNIRS) sensors, or a non-invasive neuromodulation interface.

7. The computer system of claim 6, wherein the non-invasive brainwave sensor comprises a sensor comprised of conductive polymer ink.

8. The computer system of claim 6, wherein the non-invasive brainwave sensor comprises a plurality of contact sensors affixed to a cap that is configured and disposed to be worn on the head of the non-human animal.

9. The computer system of claim 1, further comprising programming instructions that, when executed by the computer system, cause the computer system to:
receive sensor data from one or more environmental sensors; and
process the sensor data through a machine-learning system in conjunction with the non-human animal brainwave data, wherein the machine-learning system integrates multi-modal sensor fusion techniques, combining EEG, biometric, video-based pose estimation, and environmental data to refine predictive cognitive assessments.

10. The computer system of claim 1, wherein the machine-learning system includes a large language model (LLM), wherein the LLM is configured to:
perform AI-based interspecies neural translation by mapping non-human EEG signals to structured embeddings cross-referenced against species-specific behavioral lexicons;
optimize reinforcement learning-driven training sequences by dynamically adjusting behavioral conditioning techniques based on cognitive workload analysis; and
generate explainable AI-based insights using causal inference modeling, Bayesian decision-making frameworks, and hierarchical reinforcement learning strategies to improve task execution and behavioral adaptation.

11. A computer-implemented method for real-time animal training and welfare management, the method comprising executing, by a computer system having a hardware memory, software instructions stored on nontransitory machine-readable storage media to:
receive non-human animal brainwave data and biometric data;
process the non-human animal brainwave data and biometric data through a machine-learning system, wherein the machine-learning system is configured to:
isolate one or more neural patterns in the non-human animal brainwave data;
associate the one or more neural patterns with an emotional state of a non-human animal;
determine a cognitive condition based on the emotional state;
determine at least one of a training state, a welfare rating, or a predictive health condition based on the cognitive condition and biometric data;
generate an output signal indicative of the determined cognitive condition, wherein the output signal comprises at least one of an audio signal, a visual signal, a haptic signal, an olfactory signal, a neural stimulation signal, or an ultrasonic reinforcement cue;
adjust an environmental parameter based on the cognitive condition, wherein the environmental parameter comprises at least one of temperature, humidity, ambient noise level, lighting condition, or multimodal sensory enrichment;
generate a communication alert to a designated recipient based on the cognitive condition, wherein the designated recipient comprises at least one of a caretaker, veterinarian, emergency responder, operational handler, or robotic task assistant;
modify a training sequence using reinforcement learning-based adaptation, wherein the training sequence is adjusted based on real-time biometric feedback, detected cognitive workload, or reinforcement effectiveness;
translate non-human neural activity into structured communication outputs, wherein the structured communication outputs include neural embeddings mapped to an interspecies translation model; and
render and present an auditory or visual form of at least one of the cognitive condition, training state, welfare rating, predictive health condition, environmental adjustment, training adaptation, or interspecies communication output on an output device of the computer system.

12. The method of claim 11, wherein the cognitive condition includes a training state.

13. The method of claim 12, further comprising:
determining a training recommendation based on the training state, wherein the training recommendation is adapted using reinforcement learning algorithms based on real-time biometric and cognitive workload feedback; and
rendering and presenting an audio/visual form of the training recommendation on an output device of the computer system.

14. The method of claim 12, further comprising:
computing a performance metric based on the training state, wherein the performance metric is derived using AI-driven cognitive state modeling, behavioral tracking, and predictive workload estimation; and
rendering and presenting an audio/visual form of the performance metric on an output device of the computer system.

15. The method of claim 11, wherein the cognitive condition includes a welfare rating.

16. The method of claim 11, further comprising:
obtaining, via a non-invasive brainwave sensor, brainwave data from a non-human animal; and
providing the brainwave data to the computer system for processing, wherein the non-invasive brainwave sensor comprises at least one of spray-on conductive polymer inks, graphene-based EEG tattoos, functional near-infrared spectroscopy (fNIRS) sensors, or a non-invasive neuromodulation interface.

17. The method of claim 16, wherein the non-invasive brainwave sensor comprises a sensor comprised of conductive polymer ink.

18. The method of claim 16, wherein the non-invasive brainwave sensor comprises a plurality of contact sensors affixed to a cap that is configured to be worn on the head of the non-human animal.

19. The method of claim 11, further comprising:
receiving sensor data from one or more environmental sensors; and
processing the sensor data through a machine-learning system in conjunction with the non-human animal brainwave data, wherein the machine-learning system integrates multi-modal sensor fusion techniques, combining EEG, biometric, video-based pose estimation, and environmental data to refine predictive cognitive assessments.

20. The method of claim 11, wherein the machine-learning system includes a large language model (LLM), wherein the LLM is configured to:
perform AI-based interspecies neural translation by mapping non-human EEG signals to structured embeddings cross-referenced against species-specific behavioral lexicons;
optimize reinforcement learning-driven training sequences by dynamically adjusting behavioral conditioning techniques based on cognitive workload analysis; and
generate explainable AI-based insights using causal inference modeling, Bayesian decision-making frameworks, and hierarchical reinforcement learning strategies to improve task execution and behavioral adaptation.
